(19) United States
(12) Patent Application Publication
(10) Pub. No.: US 2022/0189597 A1
(43) Pub. Date: Jun. 16, 2022

(54) PERSONALIZED DIETARY SUPPLEMENT PROTOCOL AND DOSAGE FORMULATIONS

(71) Applicant: H2Yo, Parkland, FL (US)

(72) Inventors: Edgars Rozentals, Riga (LV); Alehandro Georgs Blumentals, Riga (LV); Konstantin Othmer, Los Altos Hills, CA (US)

(21) Appl. No.: 17/117,871

(22) Filed: Dec. 10, 2020

(51) Int. Cl. G16H 20/10; G16H 20/60; A23L 33/00

(52) U.S. Cl. CPC G16H 20/10; A23L 33/30; G16H 20/60

(57) ABSTRACT

A personalized dynamic dietary supplement protocol can include: analyzing health information of a subject by processing user information through a nutritional model; identifying a health condition (improvement or homeostasis) based on the nutritional condition of the subject; generating a dietary supplement protocol for the subject; determining a dosing regimen; and providing dosage formulations to the subject in data form or as physical formulations for administration of the dosage formulations to the subject in accordance with the dosing regimen to perform the dietary supplement protocol. The dosage formulations each include a temporally-personalized dosage that is formulated with a combination of the plurality of dietary supplements to provide the subject with a personalized dietary supplement regimen that dynamically personalizes delivery of each dietary supplement in each temporally-personalized dosage in accordance with the dietary supplement protocol.

### BACKGROUND

### Field

The present disclosure relates to systems and methods for designing personalized dietary supplement dosage formulations for each user. The systems and methods can provide the instructions for formulating the formulations from dietary supplement compositions, or can provide the formulated personalized dietary supplement dosage formulation for different users.

### Description of Related Art

Previously, dietary supplement protocols have been established for consumers based on predefined or stock dosages. These protocols often have a single program of supplements that are provided to a large set of consumers, if not all of the consumers. The dietary supplement protocols are often selected by the consumer to provide the consumer with supplements that the consumer wants to use.

Thus, there is a need for a technology that can be used for generating dietary supplement protocols with temporally-personalized compositions that are tailored by design for the specific consumer based on what the consumer needs in view of their health information.

### SUMMARY

In some embodiments, a method for providing a personalized dynamic dietary supplement protocol can include: inputting identification information and heath information for a subject as input data into a computing system; analyzing the health information of the subject by processing the identification information and heath information through a nutritional model to generate a nutritional condition for the subject; identifying a health condition (e.g., health improvement condition or health information improvement in subsequent health information) in the nutritional model based on the nutritional condition of the subject; generating a dietary supplement protocol for the subject to change an initial nutritional condition toward the improvement condition; determining a dosing regimen for a plurality of dietary supplements to be administered to the subject to achieve the change from the initial nutritional condition toward the health condition (e.g., homeostasis or improvement); and providing the plurality of dietary supplements in dosage formulations to the subject in data form or as physical formulations for administration of the dosage formulations to the subject in accordance with the dosing regimen to perform the dietary supplement protocol. In some aspects, the dosage formulations each include a temporally-personalized dosage that is formulated with a combination of the plurality of dietary supplements to provide the subject with a personalized dietary supplement regimen that dynamically personalizes delivery of each dietary supplement in each temporally-personalized dosage in accordance with the dietary supplement protocol.

In some embodiments, a method for providing a personalized dynamic dietary supplement protocol can include: inputting identification information and heath information for a subject as input data into a computing system; analyzing the health information of the subject by processing the identification information and heath information through a nutritional model to generate a nutritional condition for the subject; identifying a health condition in the nutritional model based on the nutritional condition of the subject; generating a dietary supplement protocol for the subject to change an initial condition toward the health condition; determining a dosing regimen for a plurality of dietary supplements to be administered to the subject to achieve the change from the initial condition toward the health condition; and providing the plurality of dietary supplements in dosage formulations to the subject for administration of the dosage formulations to the subject in accordance with the dosing regimen to perform the dietary supplement protocol. In some aspects, the dosage formulations each include a temporally-personalized dosage that is formulated with a combination of the plurality of dietary supplements to provide the subject with a personalized dietary supplement regimen.

In some embodiments, a method can include: obtaining updated health information for the subject; inputting the updated health information as updated input data into the computing system; identifying a subsequent nutritional condition after the subject is administered the dosage formulations; generating an updated dietary supplement protocol for the subject to change a subsequent nutritional condition and optionally a nutritional trajectory toward the heath condition, which can be a measurable health information improvement point; determining an updated dosing regimen for an updated plurality of dietary supplements to be administered to the subject to achieve the change from the subsequent nutritional condition toward the health condition; and providing the updated plurality of dietary supplements in updated dosage formulations to the subject for administration of the updated dosage formulations to the subject in accordance with the updated dosing regimen to perform the dietary supplement protocol. In some aspects, the updated dosage formulations each include a temporally-personalized dosage that is formulated with a combination of the plurality of dietary supplements to provide the subject with a personalized dietary supplement regimen that dynamically personalizes delivery of each dietary supplement in each temporally-personalized dosage in accordance with the dietary supplement protocol.

In some embodiments, a method for providing a personalized dynamic dietary supplement regimen can include: identifying a subject to receive a dietary supplement dosage; analyzing a dietary supplement protocol for the subject; analyzing a plurality of dietary supplement reservoir compositions to determine a mixture to provide the dietary supplement dosage; determining the dietary supplement dosage based on: the dietary supplement protocol, the plurality of dietary supplement reservoir compositions, and a dietary supplement indication, wherein the dietary supplement indication is based on at least one of: health information of the subject, a time of dosing, a dosing position in a sequence of dosings of the dietary supplement protocol, or a dietary supplement need of the subject for implementing the dietary supplement protocol; preparing the dietary supplement dosage to include a combination of the plurality of dietary supplement reservoir compositions; and providing the dietary supplement dosage to the subject for consumption.

In some embodiments, a method for providing a personalized dynamic dietary supplement regimen can include: identifying a subject to receive a dietary supplement dosage; analyzing a dietary supplement protocol for the subject; identifying a relative dosing of the dietary supplement dosage relative to other dietary supplement dosages; and determining a formulation for the dietary supplement dosage based on the identified relative dosing, wherein the formulation is formulated to provide a temporally-personalized dosage of a plurality of dietary supplements to provide the subject with a personalized dietary supplement regimen that dynamically personalizes delivery of each dietary supplement in each temporally-personalized dosage in accordance with the dietary supplement protocol.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 illustrates an embodiment of a dietary supplement dispenser that can be used with a method for providing a personalized dynamic dietary supplement protocol.

FIG. 2 shows an operational environment for the system for generating and providing the personalized dynamic dietary supplement protocol.

FIG. 3 shows a flowchart of a method of dispensing a supplement dosage formulation.

FIG. 4 shows a flowchart of a method of determining a dosing regimen for a user.

FIG. 5 includes a flowchart of a method for generating and providing personalized dietary supplement formulations.

FIG. 5A includes a flowchart of a method of providing temporally-personalized dosage.

FIG. 6 shows an example computing device (e.g., a computer) that may be used as a dispenser controller to perform the methods (or portions thereof) described herein.

FIG. 7 includes a flowchart of a method of providing temporally-personalized dosage.

FIG. 7A includes a flowchart of a method of tuning a combination of dietary supplements.

FIG. 7B includes a flowchart of a method of temporally-personalizing changed dosage formulations.

FIG. 7C includes a flowchart of a method of changing a dosing regimen.

FIG. 8 includes a flowchart of a method of providing a modulated formulation.

FIG. 9 includes a flowchart of a method of providing a supplement dosage formulation.

FIG. 10 includes a flowchart of a method of providing a supplement dosage formulation.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present technology can include a system and method for implementing a personalized supplement program that can be designed to provide a personalized dietary supplement protocol for each user. The personalized supplement program can be generated based on each user by obtaining identification information and health information for the user. The health information includes disease states, energy level, activity level, stress level, typical diet, amount of rest or sleep, any drinking of alcohol, any consumption of drugs, any smoking, pregnancy, menopause, age-related conditions, or others. The health information may also be related to the environmental conditions that the subject experiences, such as weather, pollution, pollen, ambient conditions, or the like. The identification and health information can be processed through a model or algorithm or other computation to generate a supplement protocol that is personalized for each user. The supplement protocol can be an overall plan for increasing the health or performance of the user toward an improved condition. The supplement protocol can include a regimen of one or more dosages of personalized formulations that can help the user improve their health and performance.

The personalized dosage formulations can be provided to the user for direct consumption, or the formulations can be provided as instructions for preparation provided to a dispenser that dispenses the formulation as a beverage or other consumable. The dosage formulation may or may not include water added to a supplement composition. The supplement compositions can be provided alone or in combination or with or without water for preparing the dosage formulation. The dosage formulation can be added to any type of consumable, from solid foods to liquid beverages and anything else that is consumable. In some aspects, the dosage formulations can be prepared and provided with or without a dispenser. That is, the personalized supplement program can be used with the dispensers described herein, or it could be adapted to be provided by other means described herein. The personalized supplement program can formulate and provide custom supplement formulation for each user over time. In some instances, the personalized supplement program does not provide a customized beverage based on user selection or an individual beverage level. In some instances, the personalized supplement program does not have a stock of a number of different formulations it cycles through. Instead, each formulation dispensed or otherwise provided to the user is a personalized formulation.

The personalized supplement program can include a dietary supplement protocol that includes one or more supplement dosing regimens, which further includes a plurality of individual dosage formulations. Each dosage formulation is personalized for the specific user. The personalization can be from user preference for dietary supplement and health goals. The personalization can also be from the user providing identification information and health information, which is then processed to determine the dietary supplement protocol, dosing regimens, and individual personalized dosage formulations. The user can fill out a questionnaire to provide information regarding health information, such as weight, height, age, gender, level of activity, typical diet, and the like. The health information may also be obtained passively or actively from a device of the subject or other device, such as a scale, an electronic reading of data, or an application on a smart phone. The health information may also be based on invasively obtained data from analysis of a sample from the subject. This information can be matched with their identification information (e.g., name, security number, login, etc.).

In some embodiments, the personalized supplement program can use health information that is obtained from an analysis of a biological fluid or biomarker of the subject. For example, the analysis can include proteomic, genomic, epigenomic, lipidomic, glycomic, foodomic, transcriptomics, metabolic, and others. The data can be obtained by any way possible, such as by analysis of genes, proteins, metabolites, lipids, hair, blood fluid chemistry, and the like.

In some embodiments, the personalized supplement program can use information about the user, such as historical health information. The information can be compared to new health information, such as obtained by monitoring or analyzing biological data of the user. The historical data can be compared to the monitored data (e.g., in response to a protocol), and then a dietary supplement protocol can be generated or modified based on the monitored data. Changes in the health information of a user can change subsequent dosage formulations for that user. The new health information can be obtained by biometric monitoring, such as pulse rate, respiration, temperature, blood pressure, sleep time, heart rate variability, and many others. Also, a history and current disease state or disorder can be considered, such as for example long term chronic conditions diabetes, heart, cholesterol, and many others.

In some embodiments, the input data for the subject in any of the methods can be by passive input methods. That is, the data can be collected and automatically provided to the system for use in generating the protocols or the relevant dosing formulations. Some examples of passive methods to the input and/or reading of the health condition can include: capture the height, weight, heart rate or other health indicator automatically by a supplement dispenser, such as the dispenser identifying the subject approaching the machine or after interacting with a device, such as having a picture taken stepping on a scale; or invasive methods that take a sample from the subject. The invasive methods can be biometric monitoring with those techniques listed herein as well as invasive technologies, such as any blood metrics and blood analysis means, which can include blood glucose (e.g., Continuous Glucose Meter (CGM)) as well as meters for other molecules (e.g., lactate, ketones, etc.) that use a biological sample. For example, the invasive technologies can include obtaining dialysis blood measurements to help a subject to supplement their outcomes, such as determining the subject to be low on electrolytes.

In some embodiments, the environment and season can be used for determining the dietary supplement protocol and the individual personalized formulations. For example, the day of the month or year can be used to determine a formulation, such as an immune boost during cold, flu, or covid season or outbreak. The time of day, such as morning, noon, afternoon, evening and night can be used to determine the formulation, where different times of the day can have different formulations. For example, lipid soluble supplements may be given at noon with meals but not at night, or vice versa. In another example, an afternoon formulation can give an energy boost to get the user through the afternoon. Of course, these changes area also accompanied by the overall protocol. Also, the ambient conditions, such as temperature, humidity, air quality, pollen count, source water quality, filtered water quality, or the like can be factored in determining personalized dosage formulation. Population data may also be used, where different requirements may be in rural versus urban environment.

In some embodiments, a biometer can be used for monitoring the user, such as by a smart watch, which sends the health data to the system for use in determining the dosage formulation to be delivered to the user. The biometer can monitor metrics, such as activity monitoring (e.g., steps), exercise (e.g., heart rate), sleep, work, or sitting, which can be considered when determining the personalized dosage formulation.

In some embodiments, the system can perform a study on the user by identifying a health status in one or more categories, providing specific dosage formulations over time, and assessing any changes in the health statuses. Positive improvements can be used to identify supplements or formulations advantageous to the user. Negative or declining health statuses can be used to identify supplements or formulations to void. The method is performed as a study of one user or it can be one user compared to a plurality of similar users, or even for a plurality of similar users compared to others or compared to a different group of users. The system performs a blind study on a user or a plurality of users to optimize health parameters by

Study tests supplements and combinations while monitoring health parameters and user feedback. The system automatically tunes supplements for performance parameters, such as sleep and sleep stages, speed of falling asleep, alertness, user reported awareness, and many others. This helps identify personalized formulations for each user. Additionally, the method can make comparisons between similar users in similar conditions to differentiate the effect of different protocols, different regimens, and/or different dosage formulations or different timing of dosages.

In some embodiments, the dosage formulation includes any type of dietary supplement in a suitable dosage. In some aspects, the suitable dosage is a micro-dosage. In some aspects, the micro-dosages can be spread throughout the day, such that one or more of the micro-dosages provides a daily recommended dosage. Now, a regimen, such as daily or weekly, etc., can be prepared to include the dosage formulations having the nutrients in micro-dosages to provide micronutrients. For example, a daily regimen can include a plurality of dosages each having micronutrients that are best absorbed/bioavailable in the body in smaller more frequent doses, such as vitamin C, vitamin B complex and other water soluble or lipid soluble substances. The formulations can include micronutrients such as vitamins, minerals, enzymes, amino acids, botanicals, herbs, or other dietary supplements that may be essential for enzymatic processes, biochemical processes or any other biological pathway in the fine machinery of the body (e.g., cell reproduction, energy in the cell) which is affected by hourly, daily, weekly fluctuations.

In some embodiments, the dietary supplement protocol can be determined based on the health information of the subject. The health information can generally be related to the overall health of the subject regarding their disease states, biological function, activity information, and other information that contributes to the health of a person, such as environment information, and life habit information as well as other information indicative of health.

In some embodiments, the dietary supplement protocol is configured with the dosage formulations to improve the health of the subject, which includes improving the indications related to overall health of the subject regarding their disease states, biological function, activity information, and other information that contributes to the health of a person. This may also include improving activity and life habits. In addition to improving health, the dietary supplement protocol and the dosage formulations can be configured to maintain homeostasis in the subject in one or more areas, such as those recited herein. The maintaining of homeostasis allows for the steady functioning of one or more health indicators or biological processes over a period of time or through at least one stimulus or stressful condition. That is, the dosage formulations in a regimen can be configured for homeostasis when the subject experiences a negative stimulus, such as oxidative processes typical of infection, disease, stress, exertion, as well as free radicals also triggered by pollution, allergies, weather (environmental) or the like.

In some embodiments, the nutritional condition of the subject can be related to the nutrients for health in relation to activity, stress, diet and life habits (e.g., drinking, smoking, resting, exercising, eating, etc.). The nutritional condition is an indication of the nutritional state of the subject as it relates more broadly to maintain the good functioning of body processes (e.g., homeostasis) or improving functioning of the body processes. The model can provide the nutritional condition, which is a nutritional snapshot of the health condition of the subject.

In some embodiments, the dietary supplement protocol can include providing dietary supplements to modulate biological processes for obtaining the target health condition (e.g., the health condition identified in the nutritional model based on the nutritional condition of the subject). In an example, in some cases, such as blood glucose, the target may be to reduce glucose spikes. For example, before lunch the dosage formulations can be configured to include a supplement that dampens glucose spikes for this particular subject. The system could learn over time that on certain days the subject eats more carbs/sugars than on other days, and then the system proactively doses supplements to minimize the impact of the carbs/sugars. Alternatively, the system can be configured to read the meal information that is being served to the subject from an external menu (e.g., Playground publishes the lunch menu that is obtained by the system) and then the system prepares the appropriate dosage formulation to include the proper amount of the supplement to counteract the consequences of the meal (e.g., lunch).

In some embodiments, the health condition that is identified can be an improvement condition or maintaining a condition of the subject. This allows the health condition to be a goal to be achieved in the subject by using the dosage formulations of the dietary supplement protocol. This allows the goal of the protocol to be identified, and then the dosage formulations are prepared to meet this goal. In some instance, the subject identifies some goals for a health condition by providing goal input information. In some instances, the system identifies goals for a health condition, which can be based on the identification information and health information as well as the other related information described herein, such as activity, lifestyle, environment, and the like.

In some embodiments, the goal of the health condition can be personalized for a goal specific to the subject without consideration of the population at large. That is, the goal is tailored to improve the subject specifically for something the subject needs, such as more activity, more energy, or more sleep, as well as others. In other embodiments, the goal of the health condition can be personalized in relation to the population at large. That is, the goal is tailored to improve the subject in relation to standards or averages of a given population, such as people in a geographic region, a specific gender, an age range, or others as well as combinations. Some examples of the goals can include: the same or more activity, the same or more energy, or the same or more sleep, the same or more immunity, improve a temporal imbalance, reduce stress, reduce effects of a disease state, and reduce infections, as well as others. Accordingly, the health condition to be achieved in the subject can include population goals, such as being based on a blood test, and the system determines a subject having vitamin D that is below the population average. The health condition to be achieved in the subject can include individual goals, such as subject wanting more energy or to improve sleep. In some aspects, the goals are related to achieving a similar benchmark or an improvement in heath condition biomarkers, such as heart rate variability, blood pressure, pulse, recovery rates, breathing, quality of sleep/deep sleep, blood test indicators such as Vitamin D.

In some embodiments, the health condition includes a goal to provide improvement to the subject in at least one biologically-relevant area. The improvement can be understood as reducing a gap between a current state and a desired state. For example, the goal can be to improve immunity, overcome temporal imbalances, and improve a response in the subject after a stressful activity, a disease, or an infection, as well as others.

In some embodiments, the generated dietary supplement protocol can be generated at the request of a consumer, doctor, trainer, dietician, or any other third party. In some embodiments, the generated dietary supplement protocol can be approved or selected by the consumer, doctor, trainer, dietician, or any other third party.

In some embodiments, subjects that have a specific biomarker or other indication may be more at risk of a certain disease state, and the subject may be prescribed or recommended to take a specific supplement to help reduce the risk. Accordingly, the methods described herein can also include: performing credible research on the disease state; based on the analyses described herein, recommending a supplement or supplement combination (e.g., dosing formulation) as part of the regimen and protocol; obtaining approval thereof from the consumer, doctor or other third party; and add the supplement or supplement combination to the subject's regimen. As research is updated and more is known, the system (e.g., by the server) can remotely update the formulations and recommendations provided to the user's device or to the dispenser system.

In some embodiments, the personalized dietary supplement regimen dynamically personalizes delivery of each dietary supplement in each temporally-personalized dosage in accordance with the dietary supplement protocol.

In some embodiments, the health condition is at least one of: a health condition improvement compared to an initial health condition of the subject; maintaining a health condition to be about the same as an initial health condition of the subject; an increase in energy for the subject; maintaining an energy level for the subject; an increase in activity for the subject; maintaining an activity level for the subject; increasing activity of a biological pathway in the subject; maintaining activity of a biological pathway in the subject; reducing activity of a biological pathway in the subject; increasing immunity of the subject; or maintaining immunity of the subject.

In some embodiments, the methods can include at least one of: providing information to the subject about their daily habits; providing recommendations to the subject about changing their daily habits; or personalizing the dosage formulations for the subject based on their daily habits.

In some embodiments, the health condition is a goal for the subject to achieve with the dosage formulations, wherein the goal is selected from: the same or more activity, the same or more energy, or the same or more sleep, the same or more immunity, improve a temporal imbalance, reduce stress, reduce effects of a disease state, and reduce infections, mental alertness, skin condition, bone health, joint health, digestion, glandular activity, hormonal activity, reduce inflammatory processes, aid weight management or combinations thereof. In some aspects, the health condition to be achieved in the subject includes population goals or individual goals.

In some embodiments, the methods include: tracking an amount of each dietary supplement in each dosage formulation for a subject; and saving or reporting to the subject the tracked amount of each dietary supplement for each dosage formulation or over a plurality of the dosage formulations.

In some embodiments, the personalized supplement program of the present technology relates to a dispenser that is configured to dispense different supplement compositions to formulate a supplement dosage formulation. The dispenser can dispense water with one or more different supplement compositions to formulate the dosage. The supplement compositions can be retained in reservoirs within the dispenser, and dispensed on-command with the water. Each supplement composition can be in a cartridge that is removable or replaceable in the dispenser so that they can be exchanged when empty or as desired. The dispenser can have a separate dispenser nozzle for water and for each of the different supplement compositions. Separate dispensing lines can alleviate contamination of different supplement compositions when there is residue in a line, and it gets dispensed when the composition is not supposed to.

In some embodiments, the dispenser is a retrofit device that is adapted to work with existing water coolers. The dispenser can include a water bottle adapter configured to receive a water bottle that typically goes into the water cooler base. For example, the water bottle adapter can be a recess in a top of the dispenser that is dimensioned to receive the neck of a water bottle (e.g., water service water bottle, such as 1, 2, 3, 4, 5, or more gallons). The dispenser can include a water cooler base adapter that is dimensioned as the size of a water bottle such that it fits like a water bottle into a water cooler base. That is, the dispenser fits where the water bottle usually fits, and thereby sites between the water bottle and the water cooler base. This configuration allows for the water cooler base to function to cool water and to dispense cooled water. The operations of the water cooler do not change with the retrofit dispenser. Instead, the functionality of the dispenser is added to the water cooler base.

In some embodiments, the dispenser only receives the water bottle and dispenses water and the supplement formulations. Here, the dispenser functions to dispense water with or without the supplement compositions. This dispenser includes the water bottle adapter but is devoid of the water cooler base adapter.

In some embodiments, the dispenser receives the water from an inline water source. Here, the dispenser does not include a water bottle adapter or a water cooler base adapter.

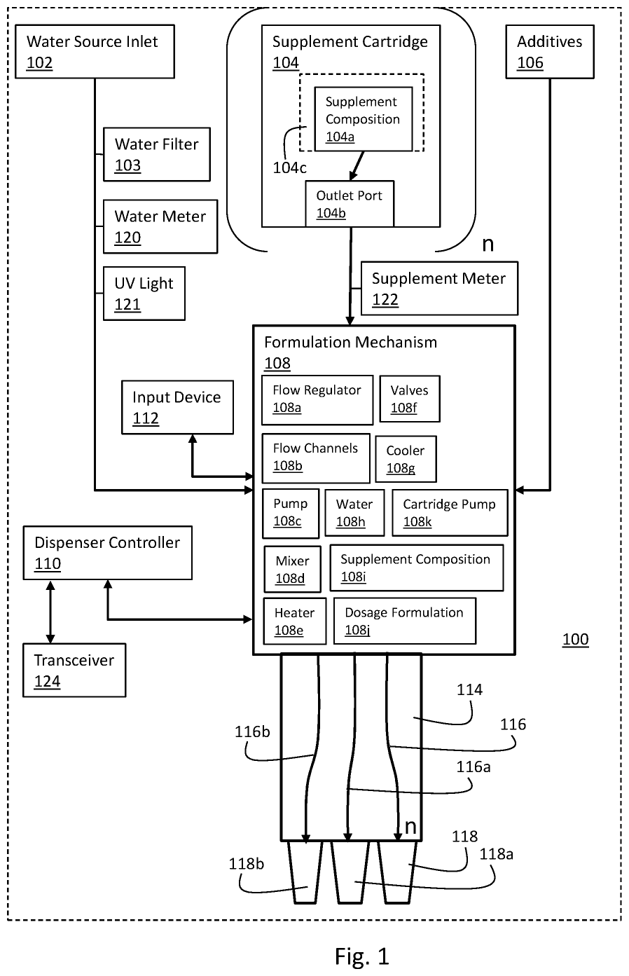

FIG. 1 illustrates a schematic diagram of an embodiment of a dietary supplement dispenser 100 that can be used to prepare and dispense the personalized supplement formulations of a personalized supplement protocol. The dietary supplement dispenser 100 includes a water source inlet 102 configured for receiving water. The water source inlet 102 can be configured to be coupled to any type of water source. For example, the water source inlet 102 can be adapted to be coupled to a water bottle (e.g., large water service bottle), a water line (e.g., water utility), a water dispenser, a water cooler, a water heater, a filtration unit, any other water source, and combinations thereof.

The dietary supplement dispenser 100 includes a plurality of supplement cartridges 104. Each supplement cartridge includes a supplement composition 104a that has one or more supplements. The supplement composition can be formulated to have supplements for a dietary supplement regimen that is adapted to provide supplemental dosage formulations to one or more different users. In some aspects, the supplement compositions can be obtained from a standard catalog of supplement compositions that can be selected or identified to fit a supplement regimen. In other aspects, the supplement compositions can be formulated for a particular user as part of a multi-composition combination. In some aspects, each cartridge includes a supplement composition 104a that is different from the other supplement compositions 104a of the other cartridges 104. The supplement composition 104a can be retained within a supplement reservoir 104c, such as a bladder or collapsible plastic or foil pouch. The cartridges 104 can include an outlet port 104b that is fluidly coupled with the supplement reservoir 104c.

The dietary supplement dispenser 100 includes at least one dispenser 114 fluidly coupled with the water source inlet 102 and plurality of supplement cartridges 104. The dispenser 114 can include a water dispenser 118a and a plurality of supplement dispensers 118b, where six supplement dispensers 118b (e.g., one for each cartridge) can be positioned around the water dispenser 118a. Each dispenser 118 can be configured with a nozzle for aiming the trajectory of the dispensed liquid. The water dispenser 118a and plurality of supplement dispensers 118b can be arranged close together to aim into the same receiver, such as a glass, mug, or other liquid container. The dispenser 114 is also shown to include flow channels 116 for water and the supplement composition 104a. The flow channels 116 can include a water flow channel 116a and a plurality (e.g., six) supplement flow channels 116b, which are fluidly coupled to the dispensers 118.

The dietary supplement dispenser 100 includes a formulation mechanism 108 that is operably coupled with the water source inlet 102 and the plurality of supplement cartridges 104. The formulation mechanism 108 includes all of the different components, pumps, valves, channels, and controllers that operate together to dispense water and the appropriate supplement composition(s) to provide a supplement dosage formulation to the user. The formulation mechanism 108 is configured for regulating fluid flow from the water source inlet 102 and the plurality of supplement cartridges 104 to the dispenser 114. The formulation mechanism includes at least one: flow regulator 108a; flow channel 108b; pump 108c; cartridge pump 108k for each supplemental cartridge 104; mixer 108d; heater 108e; valve 108f; cooler 108g; water 108h from the water source 102; supplement composition 108i from the at least one cartridge 104; and/or dosage formulation 108j as a mixture from the water and at least one supplement composition. These components can be in various arrangements and combinations to provide the water and supplement compositions 104a to the dispenser 114, such as unique fluidic pathways between each cartridge 104 or water source to the unique dispenser 118, such as water dispenser 118a or any of the different supplement dispensers 118b. In some aspects, a mixer 108d is omitted when each liquid is dispensed through a separate dispenser 118 such that there is no mixing of the supplement compositions with each other and/or with water before being dispensed from the dispenser 114.

The dietary supplement dispenser 100 includes an input device 112 configured to receive input from a user. The input device 112 can be any type of input device where the user can manually enter input data or can transmit or otherwise provide data to the dispenser 100. The input device 112 can be configured as a touch screen as shown; however, the input device 112 may include a combination of a touch screen or wireless data receiver to provide input from the user. The touch screen embodiment can be configured to provide viewable display icons for selections or a keyboard, and may display any common input display graphics with selectable options.

The dietary supplement dispenser 100 includes a dispenser controller 110 operably coupled with the formulation mechanism 108 and input device 112. The dispenser controller 110 can be configured as a computer or any computing device that has a processor that can process data and perform normal computing operations for operation of the dispenser 100 as well as receive and process input data from the input device. In addition to many operations, the dispenser controller 110 is configured to receive identification information input from a user via the input device 112. This allows the dispenser controller 110 to use the identification information of a user to access a dietary supplement protocol or particular dietary supplement regimen over a period of time to provide a customized personal supplement dosage formulation to the user for each dose. Accordingly, the dispenser controller 110 can obtain a supplement dosage formulation for the user based on a dietary supplement protocol of the user. For example, the dietary supplement protocol for the user may be stored locally in a non-transitory memory device or remotely in a database accessible over a network or through a server. Once the supplement dosage formulation for a particular dose is determined or identified, the dispenser controller 110 can control dispensing of water and at least one supplement composition 104a of the plurality of supplement cartridges 104 to provide the supplement dosage formulation to the user.

The dietary supplement dispenser 100 can include a water meter 120 operably coupled with the water source inlet 102. The water meter 120 can collect water flow data (e.g., volume, flow rate, etc.) to determine the amount of water flow used for one or more formulations, or track the water being used for one or more formulations for a specific user. The dispenser controller 110 can be configured for receiving the water meter data and monitoring water usage by at least one user and tracking water usage for the at least one user over a time period with the water meter.

The dietary supplement dispenser 100 can include at least one supplement meter 122 operably coupled with a supplement reservoir 104c having the supplement composition 104a of each supplement cartridge 104. That is, the supplement meter 122 is positioned and configured to collect supplement flow data (e.g., volume, flow rate, etc.) to determine the amount of each supplement being used, such as for one or more users. The dispenser controller 110 is configured for receiving the supplement meter data and monitoring supplement usage by at least one user and tracking supplement usage for the at least one user over a time period for each supplement composition 104a.

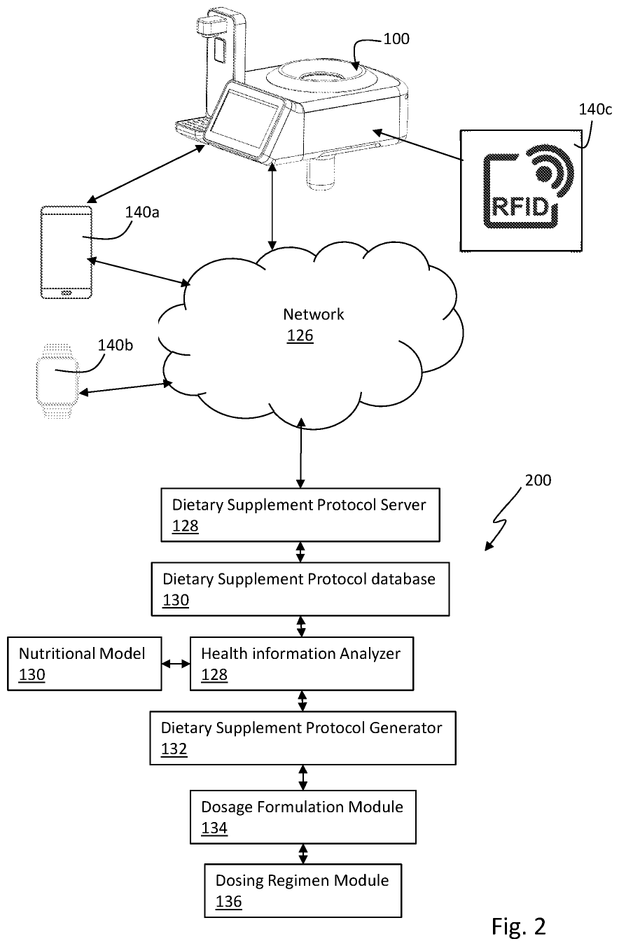

The dietary supplement dispenser 100 can include a transceiver 124 operably coupled with the dispenser controller 110 and configured to communicate over a network 126. The transceiver 124 can be any type of wireless or optical transceiver that can send data through a network (e.g., send user identification data or user water usage data or user supplement usage data) or receive data through a network (e.g., receive the supplemental dosage formulation or the dietary supplement protocol for each user. The transceiver 124 can also be used to transmit identification information for at least one user to a dietary supplement protocol server 128 and configured to receive the supplement dosage formulation of a dietary supplement protocol for the at least one user from the dietary supplement protocol server 128 (e.g., FIG. 2).

The dietary supplement dispenser 100 can include a dispenser controller 110 that is operably coupled to at least one additive reservoir 106. Each additive reservoir can have one or more additives for formulating the supplement dosage form. For example, the additive can balance pH, improve solubility or mixing of different supplements, provide for an emulsion or micelle formation, or any other additive or formulation action. The dispenser controller 110 can be configured to determine at least one additive to be included in the supplement dosage formulation and control dispensing of the at least one additive to provide the supplement dosage formulation to the user.

The dietary supplement dispenser 100 can include at last one dispenser 114 that includes at least one nozzle 118 that is configured as at least one of: a water only nozzle (e.g., 118a), a supplement only nozzle (e.g., 118b) for at least one supplement composition 104a, a supplement only nozzle for each supplement composition 104a, and a water and supplement combination nozzle.

The dietary supplement dispenser 100 can include a wireless input device 112 that is configured to receive a wireless signal from a wireless device 140 of the user. FIG. 2 shows the wireless input device 112 can be configured to communicate with a device 140a,b being a mobile phone 140a (e.g., smart phone) or a smart watch 140b. In some examples, the signal received by the input device 112 can be a signal from a mobile device, WiFi module, Bluetooth module, RFID tag 140c (e.g., FIG. 2), near field communication tag, or other signal provider designated to the user.

The dietary supplement dispenser 100 can include an input device 112 is configured to receive manual data input from the user. In some aspects, the input device 112 includes a touch screen (e.g., as shown), display, keyboard, mouse, microphone, camera, or combination thereof.

The dietary supplement dispenser 100 can include at least one water filter 103 fluidly coupled with the water source inlet 102 upstream of the formulation mechanism 108. The water filter 103 can be positioned to filter the water received from the water source inlet 102, which can be helpful if the water is municipality water or other water that may be advantageously filtered. The water filter 103 can be installed in a water filtration unit, such as those that are commonly used in water filtration applications.

The dietary supplement dispenser 100 can include a water source inlet 102 that is configured to be fluidly coupled with a water source selected from the group consisting of: a water container; a water line; a water dispenser; a water cooler; a water heater; a filtration unit; and combinations thereof. That is, the water source inlet 102 can have the proper fittings, tubes, fasteners, pumps, valves or other common water provisioning components.

The dietary supplement dispenser 100 can include a UV light 121 (e.g., UV-C light) that is configured for irradiating the water upstream of the at least one dispenser 114. That is the UV light 121 can be upstream of the formulation mechanism 108 as shown, or within the formulation mechanism 108.

The dietary supplement dispenser 100 can include a formulation mechanism 108 that has: at least one pump 108c operably coupled with the water source 102 and a water dispenser 118a of the at least one dispenser 114; a cartridge pump 108k coupled to each supplement cartridge 104 and a supplement dispenser 118b of the at least one dispenser 114; at least one water flow channel 108b; and a plurality of supplement cartridge flow channels 108b that are fluidly isolated from each water flow channel 108b.

In some embodiments, the dietary supplement dispenser 100 can include a dispenser controller 110 that is configured to control dispensing of the water and each supplement composition 104a from separate dispensers 118 so as to deliver the supplement dosage formulation to the user. This allows for the supplement dosage formulation to be formulated within the container that the dispenser 114 is dispensing into.

In some embodiments, the input device 112 includes a touch screen and is configured to receive input from a user. The dispenser controller 110 is operably coupled with the input device 112 to receive input data therefrom. The input data is used by the dispenser controller 110 to determine control instructions for controlling the formulation mechanism 108. The dispenser controller 110 is configured to: receive identification information input from a user via the touchscreen of the input device 112; obtain a supplement dosage formulation for the user based on a dietary supplement protocol of the user, which can be obtained from a local memory device of the dispenser 100 or at a server 128 or database 130 (FIG. 2); and control dispensing of water separately from each at least one supplement composition 104a of the plurality of supplement cartridges 104 to provide the supplement dosage formulation to the user.

FIG. 2 shows an operational environment for the dispenser 100 and a system 200 configured for providing personalized dynamic dietary supplement regimen. The environment shows an example of how the dispenser 100 is used with the system 200 in a method of providing a dietary supplement.

In some embodiments, the user can approach the dispenser 100 and a mobile phone 140a or a smart watch 140b may communicate with the dispenser 100 and provide identification information of the user. This can allow for the dispenser 100 to determine the supplement dosage formulation that the user should receive. Alternatively, the user can include a signal tag 140c, such as RFID or a near field communication tag that can be read by the input device of the dispenser 100, which allows the dispenser 100 to identify the user and obtain identification information of the user.

The dispenser 100 is configured with the transceiver to communicate via a network 126 to a dietary supplement protocol server 128. The server 128 can be part of a computing network that operates with the personalized dynamic dietary supplement protocol. The dietary supplement protocol server 128 can communicate with a dietary supplement protocol database 130 that has a unique dietary supplement protocol for each user. The dietary supplement protocol can include the information for preparing one or more supplement dosage formulations for a dosage regimen. The dietary supplement protocol database 130 can receive the personalized protocols from a health information analyzer 128. The heath information analyzer 128 is configured for analyzing user data, such as identification and health information, and comparing the same with a model or processed with an algorithm in facilitating the generation of the protocol. The health information analyzer 128 can be configured with an algorithm or model for analyzing the health information of the subject by processing the identification information and heath information through a nutritional model to generate a nutritional condition for the subject user. The health information analyzer 128 can be adapted for identifying a health information improvement condition in the nutritional model based on the nutritional condition of the subject. This improvement in condition can be used as the basis for the determination of the protocol. The condition may need improvement in health, disease state, vitality, stress, sleep, energy, or the like, where a need for improvement can be determined. The need for improvement can be used for determining the dosage formulations to provide to the user to get the improvement.

The health information analyzer 128 can use a nutritional model 130, such by computation with an algorithm, to determine the health condition of the subject user. Then the nutritional model 130 can identify a nutritional improvement that can provide a physiological improvement to the subject user. The nutritional improvement can then be used to determine one or more supplements for obtaining an improvement in condition in the subject user. The one or more supplements can be identified as part of a dosage formulation for being administered to the subject user, where the dosage formulation can be part of an overall dietary supplement protocol for improving the condition in the subject.

The dietary supplement protocol generator 132 can obtain the data from the health information analyzer, and then generate the dietary supplement protocol for the subject user. The generator 132 may or may not be part of the health information analyzer 128, by being in the same computational module or in separate modules. The generator 132 can be configured for receiving output data from processing with the model 130 and generating the protocol for the user subject. This can include generating the overall protocol for achieving the health improvement or maintaining the health condition. The generator 132 is configured for generating a dietary supplement protocol for the subject to change an initial nutritional condition toward the health condition. The generator 132 can be operably coupled with or include a dosage formulation module 134 and a dosing regimen module 136. The dosing formulation module 134 can be used to generate each personalized dietary supplement dosage formulation for the subject user based on the input data and processing with the nutritional model 130. The dosing regimen module 136 can be used to generate a regimen for administration of the dietary supplement dosage formulations to the subject in order to achieve the overall protocol. The dosage formulation module 134 and dosing regimen module 136 can be configured for determining a dosing regimen for a plurality of dietary supplements to be administered to the subject to achieve the change from the initial nutritional condition toward the health information improvement condition.

The dietary supplement protocol server 128 can be configured for providing the protocol, regimen, and/or personalized dosage formulations to the user, such as to the mobile phone 140a (e.g., smart phone) or smart watch 140b. The user can then provide the protocol, regimen, and/or personalized dosage formulations to a dispenser 100 for formulating and dispensing the dosage formulations to the user. Alternatively, the dietary supplement protocol server 128 can be configured for providing the protocol, regimen, and/or personalized dosage formulations to the dispenser 100, and then the dispenser 100 can dispense the dosage formulations to the user.

In some embodiments, the system can include generating, at a controller associated with a micronutrient dispenser, a nutrition dose record and a performance history update record for a person that is formulated via a health and performance algorithm and dispensed at the micronutrient dispenser, where a variety of ingredients packages contain in liquid, gel, gaseous or powder form, ingredients and mixes of ingredients that can be added to any food or drink. This method may include writing dose and performance records to a database or data processing device associated with a cohort of individuals with similar or comparable health characteristics to those of the individual being dispensed, wherein the combined record is based at least in part on the dose and performance record of the individual being dispensed. The system can be configured for dynamically regulating and complementing a user's nutritional and health profile.

In some embodiments, the controller can include at least one non-transitory memory device storing computer-executable instructions; and at least one processor communicatively coupled to the at least one memory and the display and configured to access the at least one memory and execute the computer-executable instructions to implement the methods described herein.

In some embodiments, the system includes a medical, health, or nutritional algorithm (e.g., in dispenser controller, server, or other) that produces the dosage formulation of the particular moment in time for the specific user. The formulation can be based on a history of the user's nutrition, habits, activity and performance, the sum of data of a cohort of similar individuals and their patterns of health/nutrition/supplementation practices. Also, the formulation can be based on medical, health, nutritional, and other reference values including those related to maximizing bioavailability and doses throughout the day.

In some embodiments, a dispenser can be configured as a micronutrient and supplements dispenser can include one or more of: a user interface operable to interact with a consumer and receive at least one input from the consumer (multiple health information parameters, performance, biometric, etc.); and a controller comprising a set of instructions operable to: receive, from an algorithmic dosing calculation system a dose combination of micronutrients and supplements with a variable formulation by time of day, moment in the life of the consumer.

The dietary supplement dispensers can be used in methods for providing customized and personalized dietary supplements to specific people. That is, a specific user can interact with the dispenser, such as having an account, where the dispenser determines the supplement dosage formulation that will be provided to the user during the interaction. The dispenser can obtain identification information from the user, and then acquire a dietary supplement protocol for the user. The dietary supplement protocol for that user may have a specific sequence of supplement dosage formulations that the immediate supplement dosage formulation can be selected from, or the protocol can be used for determining the immediate supplement dosage formulation based on health information of the user. The dispenser obtains the supplement dosage formulation parameters, and then determines operation protocols of the dispenser for creating the supplement dosage formulation from water with one or more of the supplement compositions in the different cartridges. The water and one or more supplement compositions can be dispensed separately into the same container (e.g., glass, cup, bottle, etc.) for the user such that the formulation is created in the container.

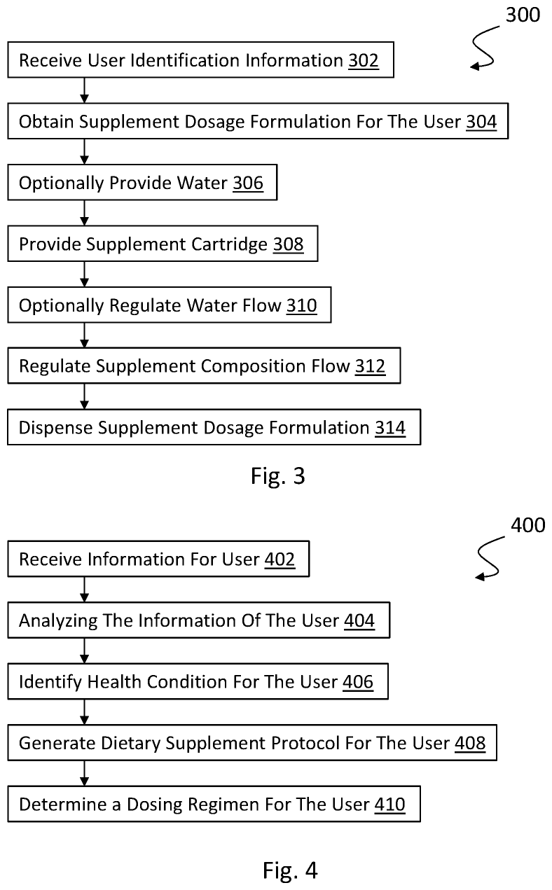

FIG. 3 illustrates a method 300 of providing a dietary supplement. The method 300 can include receiving identification information input from a user at block 302. Then, the method 300 can include obtaining a supplement dosage formulation for the user based on a dietary supplement protocol of the user at block 304. The method 300 can include providing water for the supplement dosage formulation at block 306. The method 300 can include providing a plurality of supplement cartridges at block 308, wherein each supplement cartridge includes a supplement composition that is different from the other supplement compositions of the other cartridges. The method 300 includes regulating fluid flow of the water to a water dispenser at block 310. The method 300 includes regulating fluid flow of at least one supplement composition from at least one supplement cartridge to at least one supplement dispenser at block 312. Once the formulation is determined and the flow regulations are determined, then the method 300 includes controlled dispensing of the water and the at least one supplement composition of the plurality of supplement cartridges to provide the supplement dosage formulation to the user at block 314. The supplement dosage formulation that is provided can be a specific and personalized for the user for the particular time, day, or place as well as the parameters of the protocol.

The methods can also include determining a dietary supplement protocol as well as dosing regimens and specific formulations thereof. The protocol, regimens, and formulations can be determined by the controller of the dispenser, or the determinations can be made by a remote computing system. The determinations can utilize various information about the particular user, such as their identification information and health information. The protocol can also be used as a guide or model to determine the next dietary supplement formulation to be provided to the user, such as based on current needs or sequentially relevant formulations the user has received or will receive in the future in view of the overall protocol.

FIG. 4 illustrates a method 400 of determining a dosing regimen of dietary supplement formulations for a dietary supplement protocol. The method 400 can include receiving or otherwise inputting identification information and heath information for a user as input data into a computing system at block 402. Then, the method 400 can include analyzing the health information of the user by processing the identification information and heath information through a nutritional model to generate a nutritional condition for the user at block 804. The method 400 can include identifying a health condition in the nutritional model based on the nutritional condition of the user at block 406. The method 400 can include generating a dietary supplement protocol for the user to change an initial nutritional condition toward the health information improvement condition at block 408. Once the health condition is determined, then the method 400 can include determining a dosing regimen for a plurality of dietary supplements to be administered to the user to achieve the change from the initial nutritional condition toward the health condition at block 410.

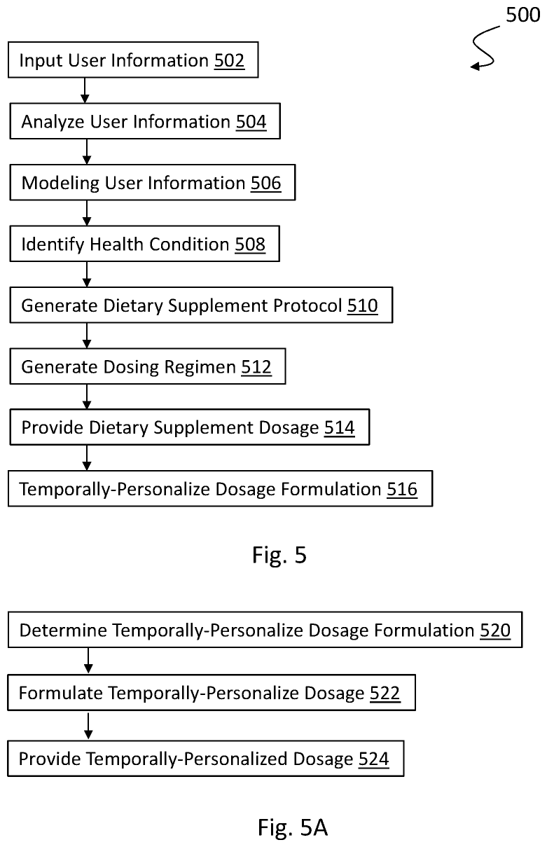

FIG. 5 illustrates a method 500 for providing a personalized dynamic dietary supplement protocol. The protocol is personalized because it is based on a subject user's identification information and health information, which together can be the user information. The protocol is dynamic because the protocol can be changed or modified based on the needs of the subject user, which allows for a dosage formulation to be generated or modified based on the needs. The everchanging nature of the dosage formulations based on needs of the subject user, as well as on the other factors described herein, creates a dynamic protocol.

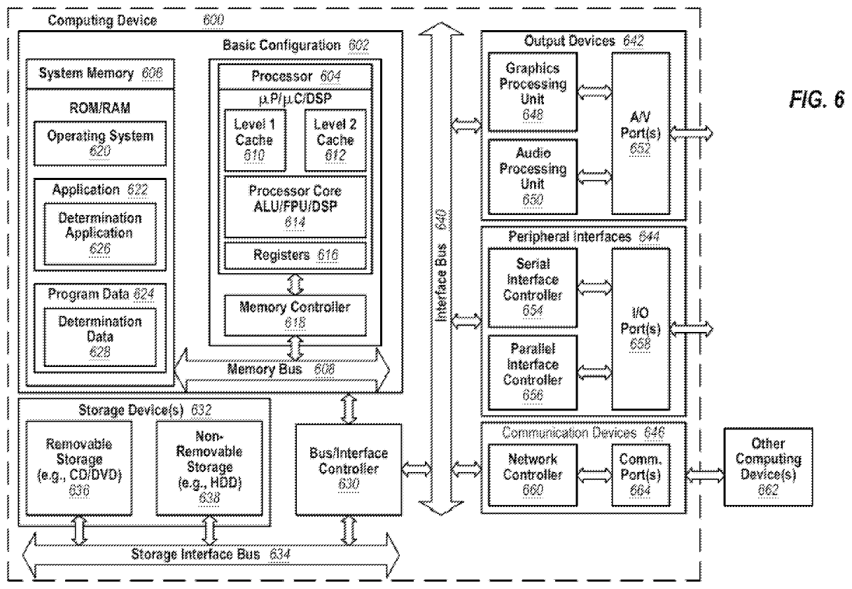

The method 500 can include inputting identification information and heath information (e.g., user information) for a subject user as input data into a computing system in block 502. The computing system is configured for performing the methods described herein such as in FIG. 6. The computing system can perform the steps of processing the user information and determining the personalized dynamic dietary supplement protocol. As such, the method 500 can include analyzing the health information of the subject at block 504, with or without the identification information. The health information can be tagged with the identification information so that the process can provide a personalized protocol. The analyzed health information can then be used by processing through a nutritional model to generate a nutritional condition for the subject at block 506. The user information can be modeled by processing the data through an algorithm for nutritional information about the nutritional condition of the subject. The method 500 can include identifying a health condition for the subject user at block 508. The health condition can be any aspect of health that can be maintained or improved, such as those described herein. The health condition can be any improvement of any condition of the subject user in any amount of improvement. The health condition can be identified in the nutritional model based on the nutritional condition of the subject. That is, the health condition that is identified can be based on the model of the subject user's health condition or nutritional condition, and such maintaining improvement can be used for identifying how to improve the subject user's health condition and nutrition condition. The method can include generating a dietary supplement protocol for the subject user to maintain or improve the health condition of the subject user at block 510. The improvement of the health condition of the subject user can be improvement from an initial nutritional condition of the subject user toward the health information improvement condition of the subject user. The protocol can be generated to provide dietary supplements to the user in order to help the user maintain or improve at least one health condition toward an improvement condition.

Once the dietary supplement protocol is generated or otherwise determined, the method 500 can include determining a dosing regimen for the subject user at block 512. The dosing regimen can be generated for a plurality of dietary supplements to be administered to the subject to achieve the health condition (e.g., maintains or improves the health information of the subject user). The health condition can result in a change from an initial nutritional condition toward the health condition. The dosing regimen can be for one or more doses of dietary supplement formulations over any duration of time. The dosing regimen may be for a time period, such as daily, weekly, biweekly, monthly, bimonthly, or even annually. During the regiment, the dynamic nature of the protocol allows for individual dosage formulations to be changed for any reason, such as based on the need of the subject or based on the relative dosing of other dosings or based on the time of day or time of year. The dosing regimen can include any number of dosage formulations that can be provided to the subject user.

The method includes providing the plurality of dietary supplements in dosage formulations to the subject user for administration in block 514. The administration of the dosage formulations to the subject are timed and/or synchronized in accordance with the dosing regimen to perform the dietary supplement protocol specifically for that subject user at block 516. The protocol can include dosage formulations that are each tailored to include a temporally-personalized dosage that is formulated with a combination of the plurality of dietary supplements. Each temporally-personalized dosage is part of a personalized dietary supplement regimen that is provided to the subject. The personalized dietary supplement regimen dynamically personalizes delivery of each dietary supplement in each temporally-personalized dosage in accordance with the dietary supplement protocol.

FIG. 5A illustrates that the method can include determining the personalized dietary supplement regimen to include a plurality of temporally-personalized dosages to be administered over a time period at block 520. Then, the method can include formulating each dosage of the plurality of temporally-personalized dosages based on the time of administration during the time period at block 522. The temporally-personalized dosage can be provided to the subject user at block 524. In some aspects, this can include temporally providing each dosage in a sequence for the plurality of temporally-personalized dosages. Also, the process of formulating each dosage of the plurality of dosages can be based on the temporal administration thereof relative to other dosages of the plurality of dosages. That is, a dosage formulation for a morning administration can be determined at least in part on the dosage formulation(s) administered the previous day and any future dosage formulation(s). Thereby, there is a temporal sequencing of dosages based on each other.

In some embodiments, the temporal administration of each dosage is based on at least one of a number of factors and considerations. The basis can be relative to an administration position of a specific personalized dosage within the personalized dietary supplement regimen. The basis can be on the health information of the subject user, which can be provided by the subject user. The basis can be on updated health information of the subject, which can be on the change in health information of the subject over time, such as from an initial time point to a current time point. The basis can be on a temporal position of the dosage relative to a year, month, day, or time of day. The basis can be on the ambient conditions of an environment where the temporal administration occurs, such as the weather conditions, including temperature, pressure, altitude, global position and any ambient conditions. The basis can be on the activity information of the subject, such as initial activity information or updated activity information based on the activity of the subject.

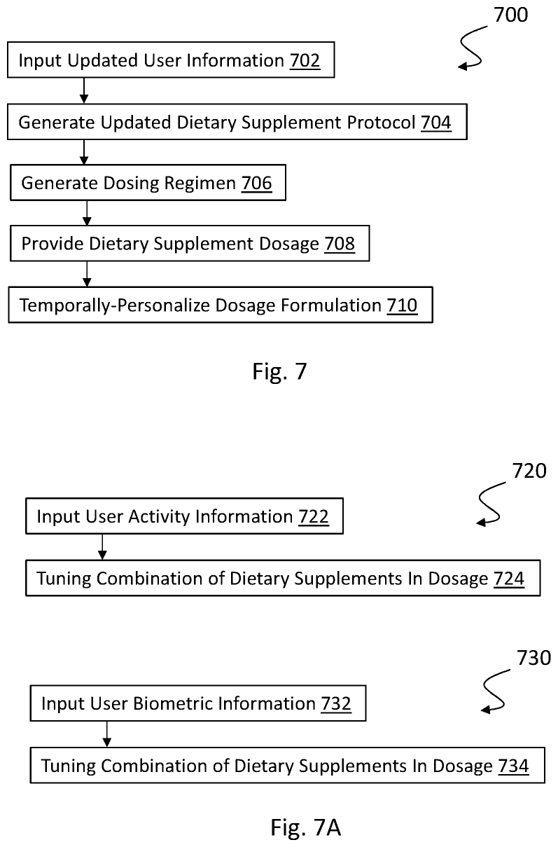

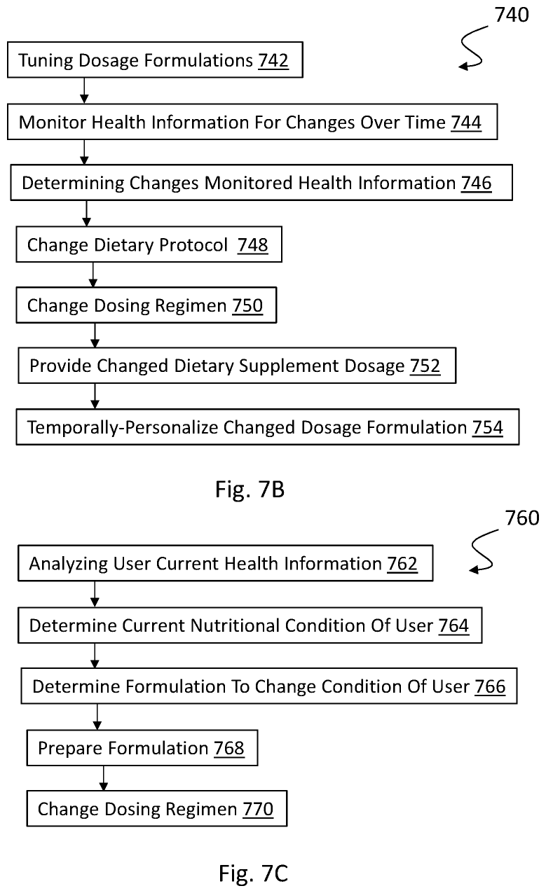

FIG. 7 shows a method 700 of updating a dietary supplement protocol by updating the specific dosage formulations and/or regimen thereof. The method 700 can include obtaining updated health information and optionally updated identification information for the subject at block 702. The information can be obtained by being input into the computing system. For example, the user can input the information either actively (e.g., into touch screen) or passively (e.g., biometric wearable) into a computing system for analysis and use in updating a protocol. The method 700 can include inputting the updated health information as updated input data into the computing system. The computing system is further configured for processing the updated health information for the methods described herein. The method 700 can include identifying a subsequent nutritional condition after the subject is administered the dosage formulations. The method 700 can include generating an updated dietary supplement protocol for the subject at block 704. The updated dietary supplement protocol can be configured to change a subsequent nutritional condition and optionally a nutritional trajectory toward the health condition or it can maintain the health condition. The method 700 can include determining an updated dosing regimen for the subject user at block 706. The updated dosing regimen can be configured for determining to combine the plurality of dietary supplements for formulations. The formulations can be configured to be administered to the subject to achieve the change from the subsequent nutritional condition and optionally nutritional trajectory toward the health condition or to maintain the health condition. The method 700 can include providing the updated plurality of dietary supplements in updated dosage forms to the subject at step 708. The dosages can be provided with instructions for administration of the updated dosage formulations to the subject in accordance with the updated dosing regimen to perform the dietary supplement protocol. The updated dosage formulations each include a temporally-personalized dosage that is formulated with a combination of the plurality of dietary supplements to provide the subject user with a personalized dietary supplement regimen that dynamically personalizes delivery of each dietary supplement in each temporally-personalized dosage in accordance with the dietary supplement protocol at block 710.

In some embodiments, the identification information includes at least height, weight, age, and gender. In some embodiments, the health information includes at least one of: level of activity; diet nutritional value; diagnosed health conditions; medications and therapeutic regimens; dietary supplement regimens; genetic information; proteomic information; biomarker information; blood pressure; pulse rate; respiration rate; blood oxygenation; perspiration; temperature; sleep information; heart condition; or combinations thereof. Other health information can also be used.

In some embodiments, the nutritional model is based on health information for a plurality of subjects and/or optimized health information for a plurality of subjects. In some embodiments, the nutritional model is a computer-generated model based on an algorithm that considers the health information of the subject in view of idealized health information for the subject or health information from a plurality of other subjects or idealized health information thereof. In some embodiments, the health condition is an optimized health condition with an improvement in measurable health information data for the subject.

In some embodiments, each of the dosage forms and/or updated dosage forms are configured with the combination of the plurality of dietary supplements for a particular administration. The particular administration can be for a single administration or a plurality of administrations for at least one of following: a date; a time of day; a day of week; a day in regimen cycle; a sequence of dosage administrations; and a relative administration in the dietary supplement protocol. In some embodiments, each of the dosage forms and/or updated dosage forms are configured with the combination of the plurality of dietary supplements for a particular administration to account for at least one of following: ambient temperature; ambient pressure; humidity; air quality index; pollution index; pollen index; source water quality; filtered water quality; weather; or climate.

In some embodiments, a method 720 as shown in FIG. 7A can include providing activity information and/or updated activity information to the computing system at step 722. The activity information and/or updated activity information is processed with the nutritional model during performance of the dietary supplement protocol. The method 720 then includes tuning the combination of the plurality of dietary supplement in at least one dose in response to the activity information and/or updated activity information at block 724.

In some embodiments, the method 730 as shown in FIG. 7A can include obtaining biometric monitored health information for the subject at one or more points during performance of the dietary supplement protocol at block 732. The biometric monitored health information is provided in real time or at different upload events. The method 730 includes tuning the combination of the plurality of dietary supplements in at least one dose in response to the biometric monitored health information at block 734.

In some embodiments, a test method 740 can be performed as shown in FIG. 7B. The test method 740 can include tuning of a dosage by changing the combination of the plurality of dietary supplement in a plurality of doses to be a plurality of study doses for a study time period at block 742. The method 740 can include monitoring health information of the subject over the study time period while taking the plurality of study doses at block 744. The method 740 can include determining changes in the monitored health information in response to the plurality of study doses at block 746.

The method 740 may also include additional steps in view of the identified changes in the health information. The method 740 can include changing the dietary supplement protocol at block 748 in response to the determined changes in the monitored health information to change a subsequent nutritional condition toward the health information improvement condition. This may include identifying a subsequent nutritional condition after the subject is administered the dosage formulations, and thereby the subsequent nutritional condition is after the initial condition. The method 740 can include determining an updated dosing regimen for an updated plurality of dietary supplements to be administered to the subject to achieve the change from the subsequent nutritional condition toward the health condition at block 750. The method 740 can include providing the updated plurality of dietary supplements in updated dosage formulations to the subject for administration of the updated dosage formulations to the subject in accordance with the updated dosing regimen to perform the dietary supplement protocol at block 752. This can include temporally-personalizing each changed dosage formulation at block 754.

In some embodiments, the methods can include using a request from a subject user to prepare a dosage formulation. This can include receiving a current dietary supplement request from the subject. Then, the method can include analyzing a current dosing regimen for the dietary supplement protocol for the subject. The system can then determine a current dietary supplement formulation with the plurality of dietary supplements in view of the current dosing regimen. The current dietary dosage formulation is then prepared and provided to the subject for consumption.

In some embodiments, the methods can include generating and administering the current dosing regimen to a subject user as shown in FIG. 7C. A method 760 can include analyzing current health information of the subject with the nutritional model at block 762. The method 760 includes determining a current nutritional condition and optionally a current nutritional trajectory of the subject at block 764. The method 760 includes determining the current dosing regimen for a current plurality of dietary supplements to be administered to the subject to achieve a change in health condition at block 766. The change in health condition can be from a current nutritional condition and nutritional trajectory toward the health condition. The method 760 can include preparing the current dietary dosage formulation to include a current combination of the current plurality of dietary supplements at block 768. The method 760 can include providing the current dietary dosage formulation to the subject for consumption at block 770.

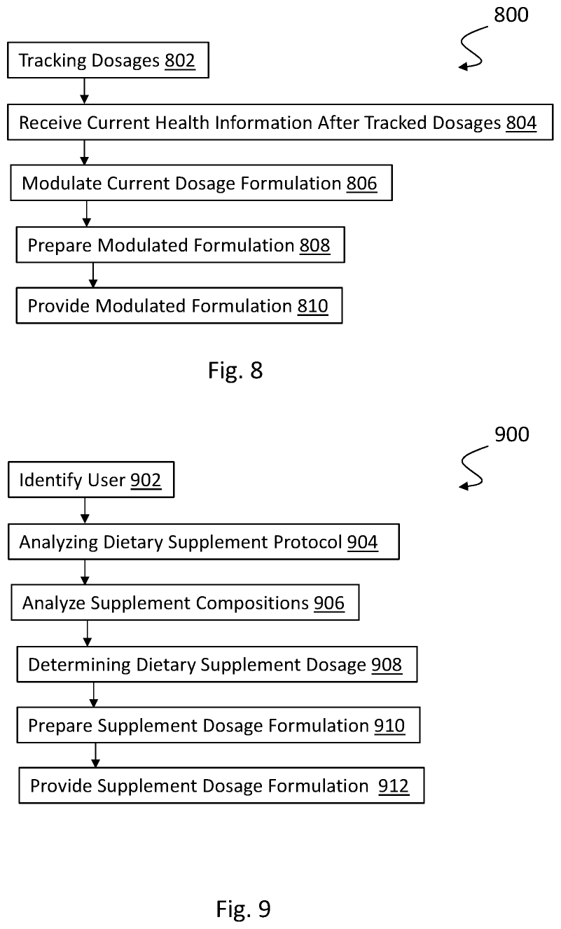

In some embodiments, a method 800 at illustrated in FIG. 8 can include tracking dosages provided to the subject in accordance with the current dosing regimen at block 802. The method 800 can include receiving the current health information in response to the tracked dosages at block 804. This may include identifying a subsequent nutritional condition after the subject is administered the dosage formulations, and thereby the subsequent nutritional condition is after the initial condition. The method 800 can include modulating the current dietary formulation at block 806. The method 800 can include preparing the modulated dietary dosage formulation to include the current combination of the current plurality of dietary supplements at block 808. The method 800 can include providing the modulated dietary dosage formulation to the subject for consumption at block 810.

In some embodiments, a method 900 for providing a personalized dynamic dietary supplement regimen can be performed with a subject user as illustrated in FIG. 9. The method 900 can include identifying the subject user to receive a dietary supplement dosage at block 902. The method 900 can include analyzing a dietary supplement protocol for the subject at block 904. The method 900 can include analyzing a plurality of dietary supplement reservoir compositions to determine a mixture to provide the dietary supplement dosage at block 906. The method 900 can include determining the dietary supplement dosage based on one or more factors at block 908 for the user. The factors can include one or more of the following: the dietary supplement protocol, the plurality of dietary supplement reservoir compositions, and a dietary supplement indication. The dietary supplement indication is based on at least one of: health information of the subject, a time of dosing, a dosing position in a sequence of dosings of the dietary supplement protocol, or a dietary supplement need of the subject for implementing the dietary supplement protocol. The method 900 can include preparing the dietary supplement dosage to include a combination of the plurality of dietary supplement reservoir compositions at block 910. The method 900 can include providing the dietary supplement dosage to the subject for consumption at block 912.

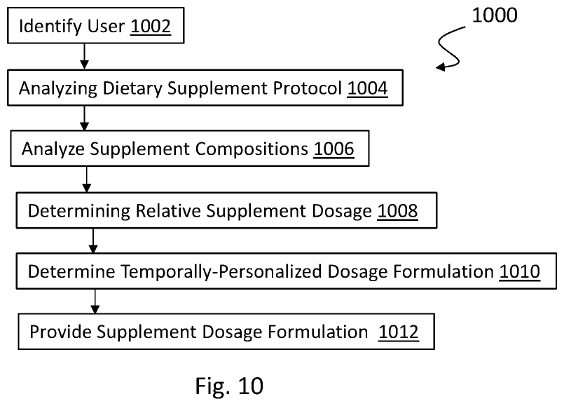

FIG. 10 illustrates a method 1000 for providing a personalized dynamic dietary supplement regimen. The method 100 can include: identifying a subject to receive a dietary supplement dosage at block 1002; analyzing a dietary supplement protocol for the subject at block 1004; identifying a relative dosing of the dietary supplement dosage relative to other dietary supplement dosages at block 1006; and determining a formulation for the dietary supplement dosage based on the identified relative dosing at block 1010. The formulation can be formulated to provide a temporally-personalized dosage of a plurality of dietary supplements to provide the subject with a personalized dietary supplement regimen that dynamically personalizes delivery of each dietary supplement in each temporally-personalized dosage in accordance with the dietary supplement protocol at block 1012.

In some embodiments, the process of identifying the subject is by at least one of: input into a device from the subject or a different person; active data from a device of the subject; passive data from a device of the subject; a signal from a device in response to a probe signal from a dosage dispenser; or passive or active acquisition of data of the subject by a dosage dispenser.

In some embodiments, the dietary supplements can be provided in reservoirs (e.g., bladders) in a cartridge, and the user can use the cartridge to dispense the dietary supplement composition. The dietary supplement composition can include one or more dietary supplement substances. The cartridges can be configured to include supplement compositions that contain vitamins and minerals that are used in numerous metabolic and chemical processes. The supplement compositions can be formulated with dietary supplements that can be used for helping the user with stress, disease, exercise, lack of sleep, ambient conditions, lack of nutrients and other factors that use vitamins and minerals in the users body. The supplement compositions can help the users' body balance physiological requirements through nourishment and can help accumulate reserves of some of these substances under many conditions.

However, it should be recognized that a user can have needs that fluctuate. The nutrition intake, ambient and activity conditions for the user can be fluctuated and can require constant attention. Food intake in normal conditions for most people requires some degree of supplementation that may not be optimally solved by the intake of large daily doses. Across a variety of vitamins and minerals the amount that can or is actually absorbed by the body varies from 10% to 90%, staying mostly well under 50%. Accordingly, the supplement compositions can be tailored and personalized for each dosage being consumed in order to alleviate any fluctuations. Each dosage can be formulated to alleviate a fluctuation in the users' needs, which can be to supplement food intake or lack of absorption of the supplements. The dosage formulation can be modified in real time to provide for increased absorption.

In some embodiments, the supplement compositions can be used for formulating personalized dosages for the user based on the user's health or health information. These personalized dosage can provide optimal bioavailability of nutrients, minerals and vitamins, which can be formulated in smaller doses, in balance with the levels of consumption imposed by stress, exercise, disease, available quality and varying nutritional intake, etc. For example, instead of one size fits all approximation of bodily requirements for nutrients, the dispenser can formulate the supplement compositions for a personalized dosage for a specific user. The dosing regimen can be configured with individual dosage formulations that are not too large, which allows for the supplements to be absorbed better instead of being lost through the digestive process and excreted, or otherwise not absorbed by the body. Thus, the dosing regimen with individual dosage formulations that are personalized can improve the uptake and health benefit of the supplements compared to bulk or generic dosing strategies for the masses.

In some embodiments, the dietary supplement protocol can be provided to a user through the digitally controlled dispensing of customized supplement dosage formulations, which are configured to include personalized individual doses of vitamins, minerals and nutrients per serving or per formulation. The personalization of each dosage formulation allows to more effectively supplement requirements for maintaining healthy body processes, ensuring the most bioavailable form of the formulated supplements. The dispenser controller or the server allows for the system to track the dosage formulations provided to the user over time. The health (e.g., health information) of the user is also tracked over the same time, which allows for a comparison on the change in health versus the dosage formulations that were provided. This provides a closed loop feedback mechanism to compare health data for a specific user over longer periods of use of the formulations. A large number of users can be served by a single dispenser or a single dispenser network (e.g., office having one or a plurality of separate dispensers) for diverse populations and large user groups, such as offices. For example, an office can have a dispenser system with a plurality of different dispensers, which allows a user to use any of the dispensers in the system to receive the dosage formulation.

The present invention provides for personalization of each dosage formulation for a user, which can be part of a dosing regimen of a dietary supplement protocol. For example, the supplement protocol can be generated or updated by obtaining information from each user regarding their physical traits as well as their activity and lifestyle. For example, every user can provide personal information input by answering questions about age, weight, height, level of activity, typical and preferred food and nutrition patterns, and also provide health information (e.g., inputs from wearable devices) such as heart rate variability, blood sugar, respiration, sleep patterns, and other health information. Any disease or disorder and state thereof can also be input. The input information allows the system to generate a dietary supplement protocol and offer supplementation in the dosage formulations that are personalized for each user at each instance of administration. This allows a suitable dispenser to provide consumable solutions that have a dietary supplement profile that best corresponds with user defined goals or needs. For example, the goals of each user can be set in the system to support immunity, daily essentials, energy levels, and similar targeted outcomes.

The present invention provides for personalized formulations for each dose to achieve high bioavailability. The design of the cartridges in the system considers formulations and preparations that allow a suitable dispenser to offer small and effective doses with minimal bodily process waste and minimal loss of nutrients through digestion or excretion. The supplement compositions (e.g., in the cartridges) use the most bioavailable chemical forms of the supplement substances. For many supplement substances there are significant differences in how much the body can absorb, which is accounted for in each personalized dosage formulation. The supplement compositions in the cartridges use the most bioavailable preparation. The system works with liquid formulations that have been shown to allow the body a much higher absorption of some substances compared to solid pills and powders for example. The supplement compositions in the cartridges use special preparation forms, such as liposomes, which ensure direct absorption into the bloodstream through the intestinal tract and avoid the degradation during the digestive processes. The supplement composition can be configured to avoid the need to observe dietary requirements, such as taking liposoluble vitamins with fats during meals. The supplement compositions in the cartridges allow their combination in such way that ensures compatibility of the components and avoid overdosing some components that accumulate in the body. The supplement compositions allow the dosing in smaller quantities throughout the day. Supplementing in the right measure, in small doses, throughout the day, optimizes the bioavailability of nutrients and reduces the workload on the body to absorb what you ingest, to keep your metabolism working at its optimal level. Accordingly, a user can be scheduled to recommended to take at least one dose, such as two, three, or four or more dosages during the day, such as from a suitable dispenser.

The supplement compositions can be designed to inhibit negative supplement interactions and/or promote positive supplement interactions with health. There are so many health aspects and nutrients users without the present invention would need considering for maintaining vital energy and a good health. Now, the present invention provides a system that assists people who are not willing to keep track of the many supplement interactions. The system can also inform personal health coaches and doctors allowing them to offer better advice to their clients. For example, an office having at least one dispenser allows the employer to provide personalized dosage formulations that are important to support immune health levels, energy and attention levels in work groups.

The supplement can include the following substances that can have interactions: Vitamin D intake effects on the immune system, bone health, hormone production and nervous system; Omega-3 effects on reducing body inflammatory processes and brain function; Vitamin B complex effects on the function of neurotransmitters, coenzymes in fat and carbohydrate metabolism; Magnesium effects in over 600 studied enzymatic metabolic reactions in the body; Selenium and Zinc functions in preventing cellular damage from free radicals. Thus, the supplements can help interrelated processes for example between gut-health and immunity

The supplement compositions can include a beneficial complementarity of herbal and other natural products. While the health claims of herbals are not always demonstrated to the same standards of vitamins and minerals, they are widely regarded as offering benefits and causing no harm. The system allows the user to incorporate supporting cartridges that may offer such complementary benefits for example for immunity and reduced inflammation levels in joints, to support the gut microbiome—the seat of our immunity.

The system is configured to monitor and track hydration for each user. The system supports and tracks the consumption of the supplements with water, therefore supporting information about the consumption of water which in itself is useful for the consumer. Thy hydration information can be health data that is used to generate or modify a dietary supplement protocol.

In some embodiments, the system can provide a multi-month program for each user that is personalized with a unique dosage formulation for each user at each administration. For example, bringing the immune system of a user to a steady low maintenance level requires supplementation for a period of time, such as through a regimen. For example, immune system cells, blood cells performing a variety of immune system functions, require 3-4 months to renew. Stores of Zinc and Vitamin D may take 1-2 months to recover high healthy levels when they are very low in a user.

In some embodiments, the system includes concentrated supplement formulations that have diverse types of supplements in combination. The supplements can include vitamins, minerals, fatty acids, antioxidants, phytonutrients, amino acids, and other nutrients carefully selected in their optimum chemical form for better bioavailability. These cartridges can include concentrated supplement formulations that are subject to personalized dosing based on each user's body, and optionally based on ambient parameters and changing daily factors in activity (under- and over exercise, mental activity), diet, rest (e.g., health information input). The system offers the user the ability to distribute smaller doses throughout the day or take all doses at once if the schedule or preferences require it, which can be performed by adjusting the amounts of supplement composition that is dispensed in each dosage formulation.

In one embodiment, the different cartridges of a dispenser system can be defined to support a well-functioning immune system with the following mixes of vitamins, minerals, fatty acids, antioxidants, phytonutrients, amino acids, and other nutrients. An example includes: Daily multivitamins and minerals (cartridge 1); Immunity boost (cartridge 2); Fat soluble (cartridge 3); and Healthy gut (cartridge 6). These different cartridge compositions are mixed and served through a regimen or supplement protocol, such as for a month long program. In another example, a performance and energy enhancing program uses a different mix focusing on the following body systems: Electrolytes (Cartridge 4); Energy enhancement (cartridge 5); and Healthy gut (cartridge 6). A significant number of bodily systems and functions can be addressed by similar programs adding a diversity of cartridges including but not limited to: Nervous system and brain function; Organ support; Heart health; Bones and joints; and Skin.

In some embodiments, a supplement composition can be configured as a daily essentials composition with vitamins and minerals. Such a daily essentials composition contains vitamins and certain minerals that are essential for everyday metabolism. The quantities are small but they are indispensable compounds, which aid in the utilization of food people consume. These compounds assist thousands of enzymes, which need vitamins and minerals for their synthesis or as cofactors in order to catalyze vital metabolic processes in human bodies. The quantities are defined in such a way that doses from this cartridge can be dispensed alone or in combinations with other cartridges. In an example, daily essentials include: B vitamins, vitamin C, folate, magnesium, selenium, zinc, iron, iodine and others. vitamin C contributes to collagen formation for the normal function of blood vessels, bones, cartilage, gums, skin and teeth. Vitamin B12, vitamin B6 and folate contribute to normal homocysteine metabolism and to normal red blood cell formation. Folate and vitamin B12 have a role in the process of cell division. Vitamin B6 contributes to the regulation of hormonal activity.

In some embodiments, a supplement composition can be configured as an immunity boosting composition. Immunity is affected by stress, insufficient and excessive exercising, unbalanced diet, gut microbiome, poor sleep, general vitamin deficit, stimulants (e.g., coffee), alcohol, chronic disease, and inflammation. An immunity boosting composition includes nutrients needed for boosting immunity so that the body can function at its optimum on a daily basis, prevent disease and to resist viral and bacterial infections. Nutrients supporting immunity include: vitamin C, vitamin D3, zinc, selenium, B vitamins, omega-3 fatty acids and others. Vitamin C contributes to maintain the normal function of the immune system, to protection of cells from oxidative stress, contributes to energy-yielding metabolism, reduction of tiredness and fatigue and functioning of the nervous system. Vitamin B12 and Vitamin B6 contribute to the normal function of the immune system.

Vitamin D contributes to the normal function of the immune system. Vitamin D has a role in the process of cell division. Vitamin D contributes to normal absorption/utilization of calcium and phosphorus, maintenance of normal bones and teeth and normal muscle function. Vitamin A contributes to the normal function of the immune system, maintenance of normal mucous membranes, skin and vision. Vitamin A has a role in the process of cell specialization.

Vitamin E contributes to the protection of cells from oxidative stress. Vitamin K contributes to normal blood clotting and to the maintenance of normal bones. DHA and EPA contribute to the normal function of the heart, to the maintenance of normal blood triglyceride levels and normal blood pressure. Coenzyme Q10 (CoQ10) is an antioxidant that our body produces naturally. Cells need CoQ10 for growth and maintenance.

In some embodiments, a supplement composition can be configured as an energy boosting composition. The fundamental source of energy in all body processes comes from special structures present in every human cell called mitochondria. Rather than relying on stimulants like caffeine the cartridge relies on micronutrients that support mitochondrial functioning. Disfunction at this level manifests through fatigue, brain fog, anxiety and depression, loss of resilience and altered sleep patterns. Mitochondria work in defense mode or energy-building mode. They cannot do both at a time. Hence a good level of supplementation aids in maintaining higher energy levels. All B vitamins contribute to normal energy-yielding metabolism. Vitamin B6, folate, vitamin B12, vitamin B2 (riboflavin), vitamin B3 (niacin), vitamin B5 (pantothenic acid) contribute to the normal functioning of the nervous system and to the reduction of tiredness and fatigue. Vitamin B1 (thiamine) contributes to normal functioning of the nervous system and to the normal function of the heart. Vitamin B2 (riboflavin) contributes to the maintenance of normal red blood cells, normal mucous membranes, skin and vision. Vitamin B2 (riboflavin) contributes to the protection of cells from oxidative stress and to the normal metabolism of iron. Vitamin B7 (biotin) contribute to the maintenance of normal mucous membranes. Vitamin B5 (pantothenic acid) contributes to normal mental performance, to normal synthesis and metabolism of steroid hormones, vitamin D and some neurotransmitters.

In some embodiments, a supplement composition can be configured as a supplement solubilizing composition. Fat soluble vitamins accumulate in the body and offer higher risks of overdosing. Intake in most forms is recommended with foods and fat intake, but in certain forms like liposomes, smaller doses can be very effectively absorbed directly in the bloodstream through the small intestine membrane. Some examples can include Vitamin D3 (cholecalciferol) and vitamin K2 (menaquinone) and omega-3 fatty acids (EPA, DHA) and coenzyme CoQ-10. For better bioavailability, these vitamins are formulated in liposomal form.

In an example, a morning formulation can include 300 ml of water and a dose of daily essentials. Then, a midday formulation can include 300 ml of water with vitamin D3, vitamin K2, and omega-3. An evening formulation can include a formulation for a sports bottle, which can include an immunity boost composition (e.g., cart. 2) and/or an energy boost composition (e.g., cart. 5).

Table 1 provides an example of cartridge compositions.

TABLE 1

| | | Cart 1 | | Cart 3 | | Cart 5 | Cart 6 |
|---|---|---|---|---|---|---|---|
| | | Daily | Cart 2 | Fat | Cart 4 | Energy | Gut |
| Element | units | essentials | Immunity | solubles | Electrolytes | boost | Health |
| C | mg | 200 | 800 | | | | |
| B12 | mcg | 100 | 100 | | | | |
| B9 Folate | mcg | 400 | 0 | | | | |
| B6 (Pyridoxine) | mg | 50 | 0 | | 75 | | |
| Mg (Malate) | mg | 200 | 0 | | 30 | 200 | |
| Se (selenomethionine) | mcg | 55 | 45 | | | 45 | |
| Zn (Orotate, Picolinate) | mg | 10 | 20 | | | 20 | |
| K (Citrate, Chloride) | mg | 50 | 0 | | | 50 | |
| Ca (Citrate) | mg | 200 | 0 | | 30 | | |
| Fe (Succinate) | mg | 10 | 0 | | | | |
| Cu (Gluconate) | mg | 1 | 1.5 | | | | |
| I (Potassium Iodite) | mcg | 100 | 50 | | | 50 | |
| Choline | mg | 100 | 0 | | | | 400 |
| (Phosphatidylcholine) | | | | | | | |
| Cr (Picolinate) | mcg | 35 | | | | | |
| D3 (Cholecaleiferol) | mcg | | | 100 | | | |
| K2 (Menaquinone-7) | mcg | | | 40 | | | |
| A (Palmitate ) | mcg | | | 900 | | | |
| E ( d-alpha tocopherol ) | mg | | | 15 | | | |
| Omega-3 | mg | | | 200 | | | |
| Ubiquinol | mg | | | 50 | | | |
| Na (from sodium chloride) | mg | | | | 80 | | |
| Cl (sodium and potassium) | mg | | | | 80 | | |
| P | mg | | | | 30 | | |
| B3 Niacin | mg | | | | | 25 | |
| B1 thiamine | mg | | | | | 5 | 5 |
| B2 riboflavin | mg | | | | | 5 | |
| B8 (Inositol NF) | mg | | | | | 100 | 500 |
| B5 Panthothenic Acid/ | mg | | | | | 5 | |
| Calcium pantothenate | | | | | | | |
| B7 (Biotin, Vit H) | mcg | | | | | 300 | |
| L-Carnitine | mg | | 0 | | 350 | 250 | 500 |
| N-Acetylcysteine | mg | | | | | 500 | |
| Methyl-Sulfonyl-Methane | mg | | | | | 1000 | |
| (MSM) | | | | | | | |

The dietary supplement protocol can be implemented with the dispensers described herein or other modes of administration. The dietary supplement protocol can be offered as a wellness program from any type of environment, such as a school, workplace, gym, home, or the like. For example, the dietary supplement protocol can be implemented in a wellness program at a place of employment, which can provide the personalized smart hydration and supplementation to provide positive health benefits and reduce health risks. The programs can be implemented to help employees improve health behavior. The program can also include instructions or operations to help people change their diets and make exercise a habit. In some aspects, the program or protocol can last 30, 60, 90, or 120 days.

In some embodiments, the system can include an application that operates on a device (e.g., 140a, 140b) of the user. The application can be configured to acquire personal information and health information from the user and provide it to the system for use in creating or updating the protocol. The application can be configured to interact with the dispenser so that the user is identified, and the dispenser formulates the correct dosage formulation for the specific user at the time the formulation is being dispensed. The application can also receive data from the system and can provide data to the user. For example, the application can be used to schedule the daily dosages of the protocol. The application can also provide alerts and notifications to the user regarding dosages. The application can also provide for data ascertainment regarding the daily activities, health, or other aspect of the user that can be used for generating or modifying a dietary supplement protocol. In some embodiments, the application can be used for tracking hydration and supplementation, which may also be tracked by the dispenser controller or the system server. In an example, the application can be used for obtaining information about the user body parameters and daily routines (e.g., exercise and diet), and providing a supplementation plan to the user. The application can track performance and activity of the user, and can correlate dosage formulations to changes in performance and activity. The application can be used to input biometrics for the user, which can be used for providing a more robust and personalized protocol. The application can also be used to transfer the personal information, health information, or dietary supplement protocol to a doctor or other care giver or health coach for the user. However, these actions can be performed by the dispenser controller or the system server.

In some embodiments, the protocols can be generated or modified by obtaining updated health information. The user can get their biological samples (e.g., blood, urine, etc.) analyzed, and then provide the health information for use in tracking the user and modifying the protocol and formulations. The health information can include: complete blood count (CBC) plus ESR; lymphocyte subpopulation tests; total antibodies—IgG, IgA, IgM; C reactive protein (CRP-hs); levels of cytokines (interleukin—6 and others); homocysteine; vitamin D; omega-3/omega-6 ratio; antioxidant mini profile; heart rate variability (HRV); biomarker analysis; or others.

In some embodiments, the dosage forms each include a temporally-personalized dosage that is formulated with a combination of the plurality of dietary supplements to provide the subject with a personalized dietary supplement regimen that dynamically personalizes delivery of each dietary supplement in each temporally-personalized dosage in accordance with the dietary supplement protocol.

In some embodiments, the methods include providing the subject with instructions for administration of the dosage formulations to the subject in accordance with the updated dosing regimen to perform the dietary supplement protocol.

In some embodiments, the system provides instructions for the dosage formulations to the user. The user can then prepare the formulations based on the instructions. For example, the user can have one or more cartridges or containers with the different supplement compositions, and the user can then prepare each formulation based on directions or recipe provided to the user. In some instance, the formulation is provided to the dispenser, and the dispenser provides the formulation to the user. In any event, the dietary supplement protocol and individual dosage formulations can be provided by the system as instructions or other data for preparation of the dosage formulations by the user or a dispenser.

The embodiments are forth in the independent claims herein. However, it should be recognized that any dependent claim from any independent claim may also depend from any other independent claim. That is, the subject matter of the dependent claims can be used with any of the methods of the independent claims or any other methods described herein.

One skilled in the art will appreciate that, for the processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In one embodiment, the present methods can include aspects performed on a computing system. As such, the computing system can include a memory device that has the computer-executable instructions for performing the methods. The computer-executable instructions can be part of a computer program product that includes one or more algorithms for performing any of the methods of any of the claims.

In one embodiment, any of the operations, processes, or methods, described herein can be performed or cause to be performed in response to execution of computer-readable instructions stored on a computer-readable medium and executable by one or more processors. The computer-readable instructions can be executed by a processor of a wide range of computing systems from desktop computing systems, portable computing systems, tablet computing systems, hand-held computing systems, as well as network elements, and/or any other computing device. The computer readable medium is not transitory. The computer readable medium is a physical medium having the computer-readable instructions stored therein so as to be physically readable from the physical medium by the computer/processor.

There are various vehicles by which processes and/or systems and/or other technologies described herein can be effected (e.g., hardware, software, and/or firmware), and that the preferred vehicle may vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The various operations described herein can be implemented, individually and/or collectively, by a wide range of hardware, software, firmware, or virtually any combination thereof. In one embodiment, several portions of the subject matter described herein may be implemented via application specific integrated circuits (ASICs), field programmable gate arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware are possible in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a physical signal bearing medium include, but are not limited to, the following: a recordable type medium such as a floppy disk, a hard disk drive (HDD), a compact disc (CD), a digital versatile disc (DVD), a digital tape, a computer memory, or any other physical medium that is not transitory or a transmission. Examples of physical media having computer-readable instructions omit transitory or transmission type media such as a digital and/or an analog communication medium (e.g., a fiber optic cable, a waveguide, a wired communication link, a wireless communication link, etc.).

It is common to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. A typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems, including feedback loops and control motors (e.g., feedback for sensing position and/or velocity; control motors for moving and/or adjusting components and/or quantities). A typical data processing system may be implemented utilizing any suitable commercially available components, such as those generally found in data computing/communication and/or network computing/communication systems.

The herein described subject matter sometimes illustrates different components contained within, or connected with, different other components. Such depicted architectures are merely exemplary, and that in fact, many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include, but are not limited to: physically mateable and/or physically interacting components and/or wirelessly interactable and/or wirelessly interacting components and/or logically interacting and/or logically interactable components.

FIG. 6 shows an example computing device 600 (e.g., a computer) that may be arranged in some embodiments to perform the methods (or portions thereof) described herein. In a very basic configuration 602, computing device 600 generally includes one or more processors 604 and a system memory 606. A memory bus 608 may be used for communicating between processor 604 and system memory 606.

Depending on the desired configuration, processor 604 may be of any type including, but not limited to: a microprocessor (μP), a microcontroller (μC), a digital signal processor (DSP), or any combination thereof. Processor 604 may include one or more levels of caching, such as a level one cache 610 and a level two cache 612, a processor core 614, and registers 616. An example processor core 614 may include an arithmetic logic unit (ALU), a floating point unit (FPU), a digital signal processing core (DSP Core), or any combination thereof. An example memory controller 618 may also be used with processor 604, or in some implementations, memory controller 618 may be an internal part of processor 604.

Depending on the desired configuration, system memory 606 may be of any type including, but not limited to: volatile memory (such as RAM), non-volatile memory (such as ROM, flash memory, etc.), or any combination thereof. System memory 606 may include an operating system 620, one or more applications 622, and program data 624. Application 622 may include a determination application 626 that is arranged to perform the operations as described herein, including those described with respect to methods described herein. The determination application 626 can obtain data, such as pressure, flow rate, and/or temperature, and then determine a change to the system to change the pressure, flow rate, and/or temperature.

Computing device 600 may have additional features or functionality, and additional interfaces to facilitate communications between basic configuration 602 and any required devices and interfaces. For example, a bus/interface controller 630 may be used to facilitate communications between basic configuration 602 and one or more data storage devices 632 via a storage interface bus 634. Data storage devices 632 may be removable storage devices 636, non-removable storage devices 638, or a combination thereof. Examples of removable storage and non-removable storage devices include: magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include: volatile and non-volatile, removable and non-removable media implemented in any method or technology for storage of information, such as computer readable instructions, data structures, program modules, or other data.

System memory 606, removable storage devices 636 and non-removable storage devices 638 are examples of computer storage media. Computer storage media includes, but is not limited to: RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 600. Any such computer storage media may be part of computing device 600.

Computing device 600 may also include an interface bus 640 for facilitating communication from various interface devices (e.g., output devices 642, peripheral interfaces 644, and communication devices 646) to basic configuration 602 via bus/interface controller 630. Example output devices 642 include a graphics processing unit 648 and an audio processing unit 650, which may be configured to communicate to various external devices such as a display or speakers via one or more A/V ports 652. Example peripheral interfaces 644 include a serial interface controller 654 or a parallel interface controller 656, which may be configured to communicate with external devices such as input devices (e.g., keyboard, mouse, pen, voice input device, touch input device, etc.) or other peripheral devices (e.g., printer, scanner, etc.) via one or more I/O ports 658. An example communication device 646 includes a network controller 660, which may be arranged to facilitate communications with one or more other computing devices 662 over a network communication link via one or more communication ports 664.

The network communication link may be one example of a communication media. Communication media may generally be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR), and other wireless media. The term computer readable media as used herein may include both storage media and communication media.

Computing device 600 may be implemented as a portion of a small-form factor portable (or mobile) electronic device such as a cell phone, a personal data assistant (PDA), a personal media player device, a wireless web-watch device, a personal headset device, an application specific device, or a hybrid device that includes any of the above functions. Computing device 600 may also be implemented as a personal computer including both laptop computer and non-laptop computer configurations. The computing device 600 can also be any type of network computing device. The computing device 600 can also be an automated system as described herein.

The embodiments described herein may include the use of a special purpose or general-purpose computer including various computer hardware or software modules.

Embodiments within the scope of the present invention also include computer-readable media for carrying or having computer-executable instructions or data structures stored thereon. Such computer-readable media can be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium which can be used to carry or store desired program code means in the form of computer-executable instructions or data structures and which can be accessed by a general purpose or special purpose computer. When information is transferred or provided over a network or another communications connection (either hardwired, wireless, or a combination of hardwired or wireless) to a computer, the computer properly views the connection as a computer-readable medium. Thus, any such connection is properly termed a computer-readable medium. Combinations of the above should also be included within the scope of computer-readable media.

Computer-executable instructions comprise, for example, instructions and data which cause a general purpose computer, special purpose computer, or special purpose processing device to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

In some embodiments, a computer program product can include a non-transient, tangible memory device having computer-executable instructions that when executed by a processor, cause performance of a method that can include: providing a dataset having object data for an object and condition data for a condition; processing the object data of the dataset to obtain latent object data and latent object-condition data with an object encoder; processing the condition data of the dataset to obtain latent condition data and latent condition-object data with a condition encoder; processing the latent object data and the latent object-condition data to obtain generated object data with an object decoder; processing the latent condition data and latent condition-object data to obtain generated condition data with a condition decoder; comparing the latent object-condition data to the latent-condition data to determine a difference; processing the latent object data and latent condition data and one of the latent object-condition data or latent condition-object data with a discriminator to obtain a discriminator value; selecting a selected object from the generated object data based on the generated object data, generated condition data, and the difference between the latent object-condition data and latent condition-object data; and providing the selected object in a report with a recommendation for validation of a physical form of the object. The non-transient, tangible memory device may also have other executable instructions for any of the methods or method steps described herein. Also, the instructions may be instructions to perform a non-computing task, such as synthesis of a molecule and or an experimental protocol for validating the molecule. Other executable instructions may also be provided.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

This application cross-references co-pending U.S. application Ser. No. 17/117,670 filed Dec. 10, 2020 and entitled “PERSONALIZED DIETARY SUPPLEMENT DISPENSING DEVICE,” which is incorporated herein by specific reference in its entirety.

All references recited herein are incorporated herein by specific reference in their entirety.

References: U.S. Pat. Nos. 3,572,553; 1,591,799; 3,250,433; 6,223,944; WO 2001079072; U.S. Pat. Nos. 9,790,079; 4,595,131; 6,196,420; 3,218,175; 3,848,776; 6,557,735; US 2005/0092769; U.S. Pat. Nos. 4,755,292; 3,060,703; 7,861,646; 6,073,539; US 2006/0000851; U.S. Pat. Nos. 5,114,047; 5,312,017; 10,464,797; 7,028,603; US 2009/0064866; U.S. Pat. Nos. 7,669,738; 9,212,041; 9,646,314; 5,390,826; US 2014/0239013; US 2006/0115570; EP 2733122; U.S. Pat. Nos. 9,668,508; 10,723,541; 10,279,985; 9,932,217; 10,231,567; 10,765,252; US 2013/0092567; U.S. Pat. Nos. 10,674,857; 8,768,524; 9,773,265; 9,051,162; 10,017,372; 10,059,581; US 2017/0099981; U.S. Pat. Nos. 7,806,294; 9,622,615; US 2006/0118581; U.S. Pat. Nos. 5,540,355; 4,958,747; 4,030,634; 5,256,279; 5,531,908; 6,793,099; 6,453,955; 6,382,467; CA 2124681; U.S. Pat. Nos. 3,363,807; and 8,728,535; US 2015/0105880; U.S. Pat. No. 8,170,405; CN 101346288; RU 2487415; U.S. Pat. No. 4,015,755; IE 47040; U.S. Pat. No. 10,694,655; US 2010/0146587; U.S. Pat. Nos. 8,309,030; 7,762,181; US 2019/0084757; U.S. Pat. Nos. 9,533,867; 10,435,285; 9,679,329; 7,438,941; and 8,606,379.

1. A method for providing a personalized dynamic dietary supplement protocol, the method comprising:
inputting identification information and heath information for a subject as input data into a computing system, wherein the computing system is configured for:
analyzing the health information of the subject by processing the identification information and heath information through a nutritional model to generate a nutritional condition for the subject;
identifying a health condition in the nutritional model based on the nutritional condition of the subject;
generating a dietary supplement protocol for the subject to change an initial condition toward the health condition; and
determining a dosing regimen for a plurality of dietary supplements to be administered to the subject to achieve the change from the initial condition toward the health condition; and
providing the plurality of dietary supplements in dosage formulations to the subject for administration of the dosage formulations to the subject in accordance with the dosing regimen to perform the dietary supplement protocol,
wherein the dosage formulations each include a temporally-personalized dosage that is formulated with a combination of the plurality of dietary supplements to provide the subject with a personalized dietary supplement regimen.

2. The method of claim 1, further comprising:
determining the personalized dietary supplement regimen to include a plurality of temporally-personalized dosages to be administered over a time period; and
formulating each dosage of the plurality of temporally-personalized dosages based on the time of administration during the time period.

3. The method of claim 2, further comprising temporally providing each dosage in a sequence for the plurality of temporally-personalized dosages.

4. The method of claim 3, further comprising formulating each dosage of the plurality of dosages based on the temporal administration thereof relative to other dosages of the plurality of dosages.

5. The method of claim 4, wherein the temporal administration is based on at least one of:
an administration position within the personalized dietary supplement regimen;
health information of the subject;
updated health information of the subject;
temporal position relative to a year, month, day, or time of day;
ambient conditions of an environment where the temporal administration occurs; or
activity information of the subject.

6. The method of claim 1, further comprising:
obtaining updated health information for the subject;
inputting the updated health information as updated input data into the computing system, wherein the computing system is further configured for:
identifying a subsequent nutritional condition after the subject is administered the dosage formulations;
generating an updated dietary supplement protocol for the subject to change the subsequent nutritional condition toward the health condition; and
determining an updated dosing regimen for an updated plurality of dietary supplements to be administered to the subject to achieve the change from the subsequent nutritional condition toward the health condition; and
providing the updated plurality of dietary supplements in updated dosage forms to the subject for administration of the updated dosage forms to the subject in accordance with the updated dosing regimen to perform the dietary supplement protocol,
wherein the updated dosage forms each include a temporally-personalized dosage that is formulated with a combination of the plurality of dietary supplements to provide the subject with a personalized dietary supplement regimen.

7. The method of claim 1, wherein:
the identification information includes at least height, weight, age, and gender; or
the health information includes at least one of: level of activity; diet nutritional value; diagnosed health conditions; medications and therapeutic regimens; dietary supplement regimens; genetic information; proteomic information; biomarker information; blood pressure; pulse rate; respiration rate; blood oxygenation; perspiration; temperature; sleep information; heart condition; or combinations thereof.

8. The method of claim 1, wherein the nutritional model is:
based on health information for a plurality of subjects and/or optimized health information for a plurality of subjects; or
a computer-generated model based on an algorithm that considers the health information of the subject in view of idealized health information for the subject.

9. The method of claim 1, wherein the health condition is an optimized health condition with an improvement in measurable health information data for the subject.

10. The method of claim 6, wherein each of the dosage forms and/or updated dosage forms are configured with the combination of the plurality of dietary supplements for a particular administration for at least one of following: a date; a time of day; a day of week; a day in regimen cycle; a sequence of dosage administrations; and a relative administration in the dietary supplement protocol.

11. The method of claim 6, wherein each of the dosage forms and/or updated dosage forms are configured with the combination of the plurality of dietary supplements for a particular administration to account for at least one of following: ambient temperature; ambient pressure; humidity; air quality index; pollution index; pollen index; source water quality; filtered water quality; weather; or climate.

12. The method of claim 1, further comprising:
providing activity information and/or updated activity information to the computing system, wherein the activity information and/or updated activity information is processed with the nutritional model during performance of the dietary supplement protocol; and
tuning the combination of the plurality of dietary supplement in at least one dose in response to the activity information and/or updated activity information.

13. The method of claim 1, further comprising:
obtaining biometric monitored health information for the subject at one or more points during performance of the dietary supplement protocol, wherein the biometric monitored health information is provided in real time or at different upload events, wherein the obtaining is active from the subject or passive from a device of the subject; and
tuning the combination of the plurality of dietary supplement in at least one dose in response to the biometric monitored health information.

14. The method of claim 1, further comprising:
changing the combination of the plurality of dietary supplement in a plurality of doses to be a plurality of study doses for a study time period;
monitoring health information of the subject over the study time period while taking the plurality of study doses; and
determining changes in the monitored health information in response to the plurality of study doses.

15. The method of claim 14, further comprising:
changing the dietary supplement protocol in response to the determined changes in the monitored health information to change a subsequent nutritional condition toward the health condition, wherein the subsequent nutritional condition is after the subject is administered initial dosage formulations;
determining an updated dosing regimen for an updated plurality of dietary supplements to be administered to the subject to achieve the change from the subsequent nutritional condition toward the health condition; and
providing the updated plurality of dietary supplements in updated dosage forms to the subject for administration of the updated dosage forms to the subject in accordance with the updated dosing regimen to perform the dietary supplement protocol.

16. The method of claim 1, further comprising:
receiving a current dietary supplement request from the subject;
analyzing a current dosing regimen for the dietary supplement protocol for the subject;
determining a current dietary supplement formulation with the plurality of dietary supplements in view of the current dosing regimen;
preparing the current dietary dosage formulation; and
providing the current dietary dosage formulation to the subject for consumption.

17. The method of claim 11, further comprising generating and administering the current dosing regimen by:
analyzing current health information of the subject with the nutritional model;
determining a current nutritional condition and optionally nutritional trajectory of the subject;
determining the current dosing regimen for a current plurality of dietary supplements to be administered to the subject to achieve the change from a current nutritional condition and optionally nutritional trajectory toward the health condition;
preparing the current dietary dosage formulation to include a current combination of the current plurality of dietary supplements; and
providing the current dietary dosage formulation to the subject for consumption.

18. The method of claim 17, further comprising:
tracking dosages provided to the subject in accordance with the current dosing regimen;
receiving the current health information in response to the tracked dosages;
modulating the current dietary formulation;
preparing the modulated dietary dosage formulation to include the current combination of the current plurality of dietary supplements; and
providing the modulated dietary dosage formulation to the subject for consumption.

19. The method of claim 1, wherein the personalized dietary supplement regimen dynamically personalizes delivery of each dietary supplement in each temporally-personalized dosage in accordance with the dietary supplement protocol.

20. The method of claim 1, wherein the health condition is at least one of:
a health condition improvement compared to an initial health condition of the subject;
maintaining a health condition to be about the same as an initial health condition of the subject;
an increase in energy for the subject;
maintaining an energy level for the subject;
an increase in activity for the subject;
maintaining an activity level for the subject;
increasing activity of a biological pathway in the subject;
maintaining activity of a biological pathway in the subject;
reducing activity of a biological pathway in the subject;
increasing immunity of the subject; or
maintaining immunity of the subject.

21. The method of claim 1, further comprising at least one of:
providing information to the subject about their daily habits;
providing recommendations to the subject about changing their daily habits; or
personalizing the dosage formulations for the subject based on their daily habits.

22. The method of claim 1, wherein the health condition is a goal for the subject to achieve with the dosage formulations, wherein the goal is selected from: the same or more activity, the same or more energy, or the same or more sleep, the same or more immunity, improve a temporal imbalance, reduce stress, reduce effects of a disease state, reduce infections, active heart rate, resting heart rate, heart rate variability, active blood pressure, resting blood pressure, recovery rate, breathing rate, quality of sleep, quality of deep sleep, and range of amount of one or more blood test indicators, or combinations thereof.

23. The method of claim 1, wherein the health condition to be achieved in the subject includes population goals or individual goals.

24. The method of claim 1, further comprising:
tracking an amount of each dietary supplement in each dosage formulation for a subject; and
saving or reporting to the subject the tracked amount of each dietary supplement for each dosage formulation or over a plurality of the dosage formulations.

25. A method for providing a personalized dynamic dietary supplement regimen, the method comprising:
identifying a subject to receive a dietary supplement dosage;
analyzing a dietary supplement protocol for the subject;
analyzing a plurality of dietary supplement reservoir compositions to determine a mixture to provide the dietary supplement dosage;
determining the dietary supplement dosage based on: the dietary supplement protocol, the plurality of dietary supplement reservoir compositions, and a dietary supplement indication, wherein the dietary supplement indication is based on at least one of: health information of the subject, a time of dosing, a dosing position in a sequence of dosings of the dietary supplement protocol, or a dietary supplement need of the subject for implementing the dietary supplement protocol;
preparing the dietary supplement dosage to include a combination of the plurality of dietary supplement reservoir compositions; and
providing the dietary supplement dosage to the subject for consumption.

26. The method of claim 25, wherein the identifying the subject is by at least one of:
input into a device;
active data from a device of the subject;
passive data from a device of the subject;
a signal from a device in response to a probe signal from a dosage dispenser; or
passive or active acquisition of data of the subject by a dosage dispenser.

27. A method for providing a personalized dynamic dietary supplement regimen, the method comprising:
identifying a subject to receive a dietary supplement dosage;
analyzing a dietary supplement protocol for the subject;
identifying a relative dosing of the dietary supplement dosage relative to other dietary supplement dosages; and
determining a formulation for the dietary supplement dosage based on the identified relative dosing, wherein the formulation is formulated to provide a temporally-personalized dosage of a plurality of dietary supplements to provide the subject with a personalized dietary supplement regimen that dynamically personalizes delivery of each dietary supplement in each temporally-personalized dosage in accordance with the dietary supplement protocol.

28. The method of claim 27, wherein the identifying the subject is by at least one of:
input into a device;
active data from a device of the subject;
passive data from a device of the subject;
a signal from a device in response to a probe signal from a dosage dispenser; or
passive or active acquisition of data of the subject by a dosage dispenser.
